(19) United States
(12) Patent Application Publication
(10) Pub. No.: US 2025/0308645 A1
(43) Pub. Date: Oct. 2, 2025

(54) ADAPTIVE MULTICOLOR REORDERING SIMULATION SYSTEM

(71) Applicants: Schlumberger Technology Corporation, Sugar Land, TX (US); Chevron U.S.A. INC., San Ramon, CA (US); TotalEnergies OneTech, Courbevoie (FR)

(72) Inventors: Christopher Aselmann-Lemon, Abingdon (GB); Hui Cao, Courbevoie (FR)

(21) Appl. No.: 18/864,024

(22) PCT Filed: May 8, 2023
(86) PCT No.: PCT/US2023/021331

§ 371 Date: Nov. 8, 2024

Related U.S. Application Data
(60) Provisional application No. 63/339,469, filed on May 8, 2022.

(51) Int. Cl. G16C 60/00; G16C 20/70

(52) U.S. Cl. CPC G16C 60/00; G16C 20/70

(57) ABSTRACT

A method can include receiving property data for a simulation model; analyzing the property data with respect to geometry of the simulation model to select a matrix preconditioner scheme using a machine model; and executing a computational simulator that implements parallel processing based on application of the matrix preconditioner scheme to a matrix representing the simulation model to generate simulation results based on the property data.

### RELATED APPLICATION

This application claims priority to and the benefit of a US Provisional Application having Ser. No. 63/339,469, filed 8 May 2022, which is incorporated herein in its entirety.

### BACKGROUND

Many complex systems in the physical sciences are studied by developing models of their underlying physics on a computing system, and by using computationally intensive methods to learn about the behavior of those complex systems. These methods are called simulations, or numerical experiments, and have applications in many fields of scientific study, from quantum chemistry to meteorology and from paleontology to the study of traffic flow patterns. Such simulations can be utilized by and/or integrated into various processes, including computerized control processes that can control equipment.

Simulations may be classified according to the type of algorithm that they employ. Discretization techniques can be utilized to transform continuous differential equations into step-by-step algebraic expressions. Monte Carlo methods can use random sampling algorithms even when there is no underlying indeterminism in a system. Cellular automata assign a discrete state to each node of a network of elements, and assign rules of evolution for each node based on its local environment in the network.

For many systems that are of interest in the computationally intensive sciences, models can be suggested directly by theory. For example, consider use of second-order, non-linear differential equations where finding useful and reliable solutions for these models, even using numerical methods, can, at times, be an unrealistic goal. Successful numerical methods, therefore, invariably demand of the simulationists that they transform the model suggested by theory substantially. A model can be substantially shaped by the exigencies of practical computational limitations and by information from a wide range of other sources.

In various instances, model equations can be analytically unmanageable under all but the most symmetric and time-independent conditions. As such, simulation demands computational methods that, given reasonable computer resources, can accurately trace out the patterns, behaviors, etc., of physical phenomena. This is not a matter of simplemindedly taking differential equations and transforming them into discrete algebraic equations. Given limitations on computing system speed and memory, these techniques may also invariably resort to other approximations, idealizations, and even “falsifications”—that is, model assumptions that may directly contradict theory. Making a simulation work, and making it produce results that the simulationist is willing to sanction as reliable, is a skill that has been developed in a lengthy period of trial, error, and comparison with both theory and known results from physical experiments.

In simulations, errors can arise as the result of transforming continuous equations into discrete ones and of transforming a mathematical structure into a computational one. Discretization techniques present the possibility of round-off errors or instabilities creating undetected artifacts in simulation results. At a deeper level, a modeling assumption that goes into creation of a simulation algorithm can have unintended consequences. Developing an appreciation for what sorts of errors are likely to emerge under what circumstances is as much an important part of the craft of the simulationists as it is of the experimenter.

A simulationist may utilize computational resources to implement one or more iterative methods for solving large sparse linear systems of equations to understand physical phenomena. Efficient iterative solvers and increased demand for solving very large systems, tend to make iterative solvers quite suitable for solving sparse linear systems.

A wide variety of iterative algorithms exist to solve sparse linear systems of equations including stationary iterative methods (e.g., Jacobi, Gauss-Seidel, Successive Over-Relaxation (SOR)), Krylov subspace methods (e.g., Conjugate Gradient (CG), Bi-Conjugate Gradient (BiCG), Generalized Minimal Residual Method (GMRES)) and Algebraic MultiGrid (AMG) methods). Krylov subspace methods and AMG methods fine use in solving sparse linear systems arising from partial differential equations (PDEs) due to their robustness and efficiency. Unfortunately, these iterative algorithms can be sequential in nature. This sequential nature results from the dependencies between computations and thereby results in increased computation time as each computation is dependent upon results from proceeding computations.

Thus, while iterative algorithms are desirable over direct solving methods because of their efficiency, the sequential nature of the computations limits performance and time saved.

An approach to improved performance of solvers involves use of a preconditioner or smoother, which can be parallel in nature thereby allowing efficient solving of systems of equations using iterative methods. For example, consider a multi-color diagonal-based incomplete lower unitriangular, upper triangular (DILU) preconditioner that is suitable for implementation on a parallel hardware architecture (e.g., GPU hardware) as set forth in U.S. Pat. No. 9,798,698, entitled “System and method for multi-color DILU preconditioner”, which is incorporated by reference herein in its entirety. Such an approach can utilize coloring to extract parallelism in a DILU smoother or preconditioner where, for example, a system may be operable to perform multi-color DILU preconditioning in parallel thereby providing enhanced performance over existing ILU preconditioners, which tend to be difficult to parallelize. Parallelism can allow faster completion of preconditioning or smoothing over sequential methods. In various instances, a multi-color DILU preconditioner may demand relatively low storage and be relatively inexpensive computationally.

A preconditioning method can include, for example, accessing a matrix that includes a plurality of coefficients of a system of equations and accessing coloring information corresponding to the matrix. In such an approach, the method can include determining a diagonal matrix based on the matrix and the coloring information corresponding to the matrix where, for example, determining of the diagonal matrix may be determined in parallel on a per color basis, for example, using a parallel hardware architecture (e.g., a graphics processing unit (GPU), multiple cores, multiple CPUs, etc.).

While a particular preconditioner, or smoother, is mentioned, one or more other preconditioners, or smoothers, may be utilized, additionally or alternatively. Further, while a particular coloring technique is mentioned, one or more other coloring techniques may be utilized, additionally or alternatively. To expedite a simulation workflow, demands on at least some decisions made by humans as to techniques may be facilitated and/or made by a machine. In such an approach, decisions and simulations, including simulation results, may be more consistent, reliable, comparable, etc. Such an approach may aim to capture aspects of the art of simulation by one or more machine models that can be executed efficiently to improve simulation.

### SUMMARY

A method can include receiving property data for a simulation model; analyzing the property data with respect to geometry of the simulation model to select a matrix preconditioner scheme using a machine model; and executing a computational simulator that implements parallel processing based on application of the matrix preconditioner scheme to a matrix representing the simulation model to generate simulation results based on the property data.

A system can include a processor; a memory accessible by the processor; and processor-executable instructions stored in the memory that are executable to instruct the system to: receive property data for a simulation model; analyze the property data with respect to geometry of the simulation model to select a matrix preconditioner scheme using a machine model; and execute a computational simulator that implements parallel processing based on application of the matrix preconditioner scheme to a matrix representing the simulation model to generate simulation results based on the property data.

One or more non-transitory computer-readable storage media can include computer-executable instructions executable to instruct a computer to: receive property data for a simulation model; analyze the property data with respect to geometry of the simulation model to select a matrix preconditioner scheme using a machine model; and execute a computational simulator that implements parallel processing based on application of the matrix preconditioner scheme to a matrix representing the simulation model to generate simulation results based on the property data.

This summary is provided to introduce a selection of concepts that are further described below in the detailed description. This summary is not intended to identify key or essential features of the claimed subject matter, nor is it intended to be used as an aid in limiting the scope of the claimed subject matter.

### BRIEF DESCRIPTION OF THE DRAWINGS

Features and advantages of the described implementations can be more readily understood by reference to the following description taken in conjunction with the accompanying drawings.

FIG. 1 is a diagram of an example of a system and an example of a geologic environment.

FIG. 2 is a diagram of an example of a sedimentary basin and an example of a system.

FIG. 3 is a diagram of an example of a method.

FIG. 4 is a diagram of an example of a method.

FIG. 5 is a diagram of an example of a machine model.

FIG. 6 is a diagram of an example of a portion of a simulation model.

FIG. 7 is a diagram of an example of a simulation model.

FIG. 8 is a diagram of an example of a simulation model.

FIG. 9 is a diagram of an example of a GPU hardware architecture.

FIG. 10 is a series of diagrams of example plots.

FIG. 11 is a diagram of an example of a framework.

FIG. 12 is a diagram of an example of a method and an example of a system.

FIG. 13 is a diagram of an example of a geologic formation with a well and an example of a matrix.

FIG. 14 is a diagram of an example of a data structure with color-coding.

FIG. 15 is a diagram of an example of a method.

FIG. 16 is a diagram of example components of a system and a networked system.

### DETAILED DESCRIPTION

The following description is not to be taken in a limiting sense, but rather is made merely for the purpose of describing the general principles of the implementations. The scope of the described implementations should be ascertained with reference to the issued claims.

As explained, simulation can be utilized to understand various types of physical phenomena where one or more types of numerical techniques may be implemented. Numerical techniques can involve discretization of equations, spatially and/or temporally. Numerical techniques can include meshless techniques (or meshfree techniques), finite element techniques, finite difference techniques, finite volume techniques, amongst other techniques. Various techniques involve cells, nodes, meshes, grids, etc. For example, a meshless technique can be free of connections between nodes of a simulation domain and rely on interaction of each node with its neighbors.

Meshless/meshfree techniques find use as alternatives to techniques such as the finite element method, especially for problems involving material failure, dynamic fracture and fragmentation, large deformations, and complex volumetric domain parametrizations. For example, consider the smoothed particle hydrodynamics (SPH) method, which belongs to one of the earliest meshless methods and was first applied to problems in astrophysics. In some instances, the computational cost of SPH simulations per number of particles can be substantially larger than the cost of grid-based simulations per number of cells when the metric of interest is not (directly) related to density (e.g., the kinetic-energy spectrum). Therefore, if one overlooks issues of parallel processing speedup, simulation of constant-density flows (e.g., external aerodynamics) tends to be more efficient with grid-based methods than with SPH.

In various examples, a preconditioner scheme may be applied for a numerical technique that involves nodes that move, whether the nodes are particles, grid nodes, etc. For example, an inverse finite element method can solve for positions of isotherms, streamlines, boundaries, implicit function values, etc. In such an example, an initial discretization, or an intermediate discretization, may be utilized and assessed for purposes of preconditioner scheme selection.

Whether discretized in space and/or time, node-to-node (e.g., or center-to-center) distances may be constant, variable, symmetric, asymmetric, etc. For example, in the field of reservoir simulation, discretization may be finer along a depth dimension than along a lateral dimension, whether in a Cartesian coordinate system space, a cylindrical coordinate system space, etc. In the field of reservoir simulation, discretization may be driven by data (e.g., seismic data indicative of subsurface structures, downhole log data, etc.), physical structures (e.g., faults, geobodies, etc.) and/or by physical phenomena (e.g., gradients, etc.). As an example, an aspect ratio or aspect ratios may be utilized to characterize a discretization. For example, consider an aspect ratio for node spacings, an aspect ratio for cell sizes/shapes, etc. In some instances, limits may be placed on aspect ratio, for example, where “skinny” cells may give rise to numerical computational issues related to physical phenomena.

In reservoir simulation, physical phenomena studied can include fluid flow. For example, consider a reservoir that can be characterized to generate a model of the reservoir. Such a model can incorporate various characteristics of the reservoir such as, for example, those that are pertinent to its ability to store hydrocarbons and also to produce them. A reservoir characterization model may be utilized for simulation of behavior of fluids within the reservoir under different sets of circumstances and to find the optimal production techniques that will maximize the production.

Characteristics of a reservoir can include permeability, amongst other characteristics. Permeability is the ability, or measurement of a rock's ability, to transmit fluids, typically measured in darcies or millidarcies (e.g., consider Darcy's law or the Darcy equation, which stem from a demonstration that heat transfer equations may be modified to adequately describe fluid flow in porous media). Formations that transmit fluids readily, such as sandstones, can be described as being permeable as they tend to have many large, well-connected pores. Other types of formations may be defined as being impermeable formations, such as shales and siltstones, which tend to be finer grained or of a mixed grain size, with smaller, fewer, or less interconnected pores. Absolute permeability is the measurement of the permeability conducted when a single fluid, or phase, is present in the rock. Effective permeability is the ability to preferentially flow or transmit a particular fluid through a rock when other immiscible fluids are present in the reservoir (e.g., effective permeability of gas in a gas-water reservoir). The relative saturations of the fluids as well as the nature of the reservoir can affect effective permeability. Relative permeability is the ratio of effective permeability of a particular fluid at a particular saturation to absolute permeability of that fluid at total saturation. If a single fluid is present in a rock, its relative permeability is 1.0. Calculation of relative permeability allows for comparison of the different abilities of fluids to flow in the presence of each other, as the presence of more than one fluid generally inhibits flow.

A characteristic related to permeability is transmissibility, which can be a measure of conductivity of a formation as to fluid. Transmissibility may be defined by a particular simulator in a particular manner. As an example, transmissibility may be defined with respect to viscosity (e.g., adjusted for viscosity, etc.). For example, transmissibility can be a measure of the conductivity of a formation adjusted for viscosity of flowing fluid. Another characteristic related to permeability is mobility, which is the ratio of effective permeability to phase viscosity. As to mobility, it may be controlled, for example, using foaming agents (e.g., surfactants, etc.). For example, consider a foaming agent as a viscosity-enhancing mobility-control agent that can be injected via an injection well to improve production form a production well. Simulation of injection and production can generate results that can guide planning, execution and/or control of field operations.

As to an example of a simulator, consider an integrated flow simulator referred to as the IFLO simulator, which is an iterative, implicit pressure-explicit saturation finite difference simulator. For the IFLO pseudomiscible, multicomponent, multidimensional fluid flow simulator, isothermal, Darcy flow can be modeled in up to three dimensions. The IFLO simulator assumes reservoir fluids can be described by up to three fluid phases (oil, gas, and water) with physical properties that depend on pressure and composition. Natural gas and injected solvent are allowed to dissolve in both the oil and water phases. A feature of the IFLO simulator provides for integration of a petrophysical model with a flow simulator. For example, an integrated flow model can provide for acoustic velocity and impedance calculations. Such reservoir geophysical calculations make it possible to track changes in seismic variables as a function of time, which can be a basis for 3D time-lapse (4D) seismic analysis. The IFLO simulator defines transmissibility for flow between neighboring gridblocks as a series application of Darcy's law. For example, a transmissibility term can be defined between two gridblocks using the product of average values of relative permeability of a phase, absolute permeability of each gridblock at the interface and cross-sectional area of each gridblock at the interface divided by the product of viscosity of the phase and the formation volume factor of the phase in each gridblock. The transmissibility to each phase can be determined using a harmonic average calculation of the product of absolute permeability times cross-sectional area at the interface between neighboring gridblocks. An arithmetic average of phase viscosities and formation volume factors may be utilized. The average relative permeability can be determined using an upstream weighted averaging technique. In the IFLO simulator, fully implicit formulations can update relative permeability, viscosity and formation volume factor as pressure and saturation distributions change during iterative computations that occur within a timestep. As an example, a solver of a simulator may implement an implicit pressure, explicit saturation (IMPES) scheme. Such a scheme may be considered to be an intermediate form of explicit and implicit techniques. In an IMPES scheme, saturations are updated explicitly while pressure is solved implicitly. IMPES formulations can update relative permeability, viscosity, and formation volume factor using new pressure and saturation distributions following completion of a timestep. In a model, at particular interfaces, transmissibility may be set to zero to represent a no-flow boundary condition (e.g., consider a side of a domain, a sealing fault, etc.).

As an example, a simulator may utilize transmissibility when performing simulation runs as to fluid movement in a reservoir and/or equipment (e.g., conduits, etc.) operatively coupled to the reservoir (e.g., via fluid communication, etc.).

As explained, for reservoir simulation, transmissibility depends on permeability and model geometry. One or more other types of simulations may include one or more characteristics that also depend on model geometry. Where a characteristic depends on model geometry, that characteristic may be utilized for improving a simulation workflow, for example, by using a machine model that can make one or more decisions.

As explained, a reservoir simulator can be used to enable reservoir engineers to understand the behavior of hydrocarbon reservoirs to enable them to plan their operation with best efficiency. A reservoir simulator can include a solver that solves a set of partial differential equations, for example, using an implicit finite volume scheme or another type of scheme.

In linear algebra and numerical analysis, a preconditioner P of a matrix A is a matrix such that P-1A has a smaller condition number than A; noting that preconditioning can involve multiplication of a column vector, or a block of column vectors, by a preconditioner, which may be performed in a so-called matrix-free fashion. For example, a so-called matrix-free method can include an algorithm for solving a linear system of equations or an eigenvalue problem that does not store the coefficient matrix explicitly, but accesses the matrix by evaluating matrix-vector products. Such an approach can be preferable at times, for example, when the matrix is so big that storing and manipulating it would involve considerable memory demands and computing time, even with use of methods for sparse matrices.

Preconditioners tend to be useful in iterative methods to solve a linear system Ax=b for x since the rate of convergence for most iterative linear solvers increases because the condition number of a matrix decreases as a result of preconditioning; noting that a large condition number can be indicative of a matrix being ill-conditioned. Preconditioned iterative solvers can outperform direct solvers (e.g., Gaussian elimination), for large, sparse, matrices. As mentioned, iterative solvers can be used as so-called matrix-free methods, where a coefficient matrix A is not stored explicitly (e.g., rather accessed by evaluating matrix-vector products, etc.).

Various numerical solvers may be classified as being explicit or implicit. When a direct computation of dependent variables can be made in terms of known quantities, the numerical technique can be referred to as explicit; whereas, when the dependent variables are defined by coupled sets of equations, and either a matrix or iterative technique is demanded to obtain a solution, the numerical technique can be referred to as implicit.

As an example, consider computational fluid dynamics where governing equations tend to be nonlinear and where the number of unknown variables can be quite large. In such circumstances, implicitly formulated equations tend to be used and solved using iterative techniques.

Iterations are used to advance a solution through a sequence of steps from a starting state to a final, converged state. This is true whether the solution sought is either one step in a transient problem or a final steady-state result. In either case, the iteration steps resemble a time-like process; noting that time can be advanced after each result. For example, consider time be discretized using a suitable numerical technique to advance from one result at one time to another result at another time.

As an example, an implicit solver can rely on a heuristically modified Newton solver, which in turn demands that the linear problem Ax=b is solved for x repeatedly, where A is a known matrix, x is an unknown vector and b is a known vector (e.g., noting that so-called matrix-free techniques may be utilized). Solving such a system of equations efficiently in a highly parallel computational environment can be challenging. As explained, a preconditioner or a smoother may be implemented such as, for example, a preconditioned Krylov method (flexible generalized minimal residual method or FGMRES). Such a preconditioner can have multiple stages, where an incomplete LU factorization can be employed in one of the multiple stages. FGMRES relates to the generalized minimal residual method (GMRES), which is an iterative method for the solution of large linear systems of equations. However, GMRES does not always perform well when applied to the solution of linear systems of equations that arise from the discretization of linear ill-posed problems with error-contaminated data represented by the right-hand side. Such linear systems are commonly referred to as linear discrete ill-posed problems. The FGMRES method is a generalization of GMRES that allows larger flexibility in the choice of solution subspace than GMRES.

As an example, to allow fine grained parallelism, which is particularly useful for GPU devices, the approach described in U.S. Pat. No. 9,798,698 for a multicolor reorder preconditioner, can be refined by context specific further approximation and re-ordering to improve performance.

If applied to systems of equations as in the field of reservoir simulation, a multicolor reordered incomplete LU preconditioner can lead to poor convergence in many difficult simulations. This is caused by problems with the specific color reordering employed, which is not suitable for the range of parameters and features encountered in reservoir simulations. The convergence can be improved by increasing the number of colors, but this reduces the efficiency of the technique.

As an example, a system can include features for improving a multicolor approach by identifying particular parameters and features, designing alternative coloring schemes to handle the different parameter ranges and features, and automatically selecting an acceptable coloring scheme. As an example, a preconditioner can be a modified multicolor reorder preconditioner that is also modified to allow some terms to be dropped to maintain high efficiency for parallel processing, in particular, for GPU devices. Various trials demonstrate substantial performance improvements over the multicolor reorder preconditioner.

As to preconditioners and parallel techniques, an article by Fung et al., “Parallel Unstructured-Solver Methods for Simulation of Complex Giant Reservoirs.” SPE J. 13 (2008): 440-446. doi: https://doi.org/10.2118/106237-PA, is incorporated by reference herein in its entirety.

As explained, a system can include features for implementing one or more coloring schemes devised to handle specific parameter ranges and features in a simulation. Such schemes can provide improved convergence and efficiency, compared to various existing techniques with respect to a particular subset of models. For example, various models include a structured grid that has stronger transmissibility in the vertical direction, and which is further complicated by faults (e.g., sealing faults, etc.). Other models might include local grid refinements which complicate structure simplicity. By tailoring each coloring algorithm to handle a particular scenario more effectively, a system can improve performance. To select the most suitable scheme for an arbitrary simulation model, a system can include an adaptive selector.

As an example, an adaptive selector can operate via an action that involves examining a simulation discretization, which may be a grid, a mesh, nodes, etc. For example, in the field of reservoir simulation, average transmissibility can be computed in vertical columns and in areal planes where the ratio of these two quantities can be stored to memory (e.g., in a memory device). In such an example, if there are local grid refinements present, this information can also be stored to memory by the system. Depending on this information, a machine model can be implemented to select from a group of different coloring schemes, and to select an appropriate number of colors for a selected scheme. For example, consider use of a decision tree as a machine model that can be used to select from a group of different coloring schemes and to select an appropriate number of colors for a selected scheme. While a decision tree is mentioned, one or more other machine models, data structures, etc., may be utilized, additionally or alternatively.

Before describing various examples in more detail, in the field of subsurface simulations, consider various computational frameworks, types of equipment, etc., that may be utilized for performing simulations, for performing field operations, etc.

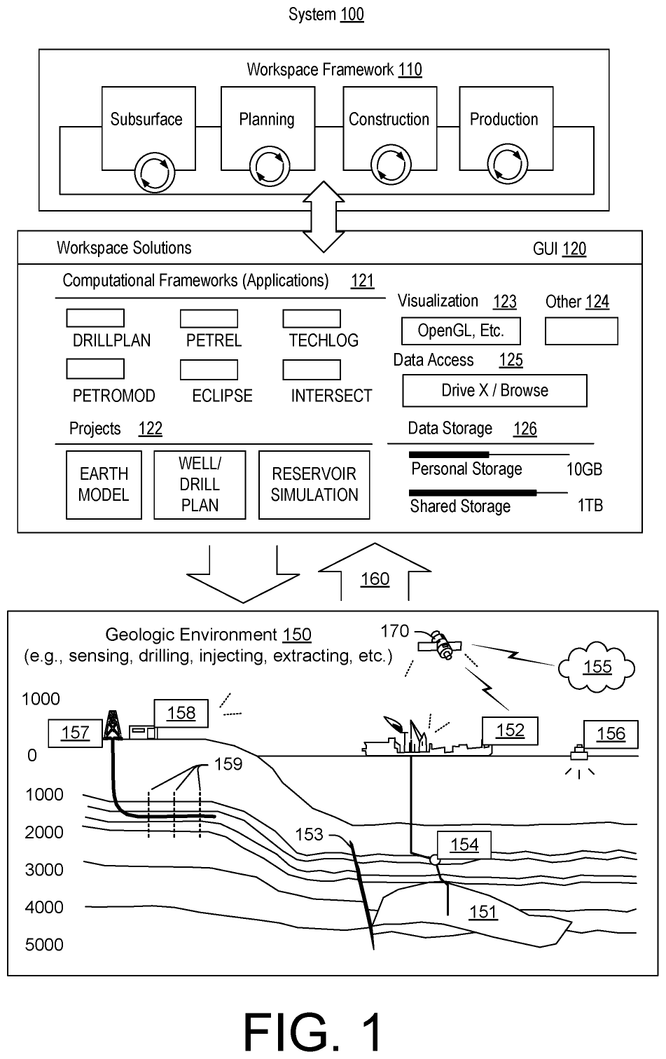

FIG. 1 shows an example of a system 100 that includes a workspace framework 110 that can provide for instantiation of, rendering of, interactions with, etc., a graphical user interface (GUI) 120. In the example of FIG. 1, the GUI 120 can include graphical controls for computational frameworks (e.g., applications) 121, projects 122, visualization 123, one or more other features 124, data access 125, and data storage 126.

In the example of FIG. 1, the workspace framework 110 may be tailored to a particular geologic environment such as an example geologic environment 150. For example, the geologic environment 150 may include layers (e.g., stratification) that include a reservoir 151 and that may be intersected by a fault 153. As an example, the geologic environment 150 may be outfitted with a variety of sensors, detectors, actuators, etc. For example, equipment 152 may include communication circuitry to receive and to transmit information with respect to one or more networks 155. Such information may include information associated with downhole equipment 154, which may be equipment to acquire information, to assist with resource recovery, etc. Other equipment 156 may be located remote from a wellsite and include sensing, detecting, emitting or other circuitry. Such equipment may include storage and communication circuitry to store and to communicate data, instructions, etc. As an example, one or more satellites may be provided for purposes of communications, data acquisition, etc. For example, FIG. 1 shows a satellite 170 in communication with the network 155 that may be configured for communications, noting that the satellite may additionally or alternatively include circuitry for imagery (e.g., spatial, spectral, temporal, radiometric, etc.).

FIG. 1 also shows the geologic environment 150 as optionally including equipment 157 and 158 associated with a well that includes a substantially horizontal portion that may intersect with one or more fractures 159. For example, consider a well in a shale formation that may include natural fractures, artificial fractures (e.g., hydraulic fractures) or a combination of natural and artificial fractures. As an example, a well may be drilled for a reservoir that is laterally extensive. In such an example, lateral variations in properties, stresses, etc. may exist where an assessment of such variations may assist with planning, operations, etc. to develop a laterally extensive reservoir (e.g., via fracturing, injecting, extracting, etc.). As an example, the equipment 157 and/or 158 may include components, a system, systems, etc. for fracturing, seismic sensing, analysis of seismic data, assessment of one or more fractures, etc.

In the example of FIG. 1, the GUI 120 shows some examples of computational frameworks, including the DRILLPLAN, PETREL, TECHLOG, PETROMOD, ECLIPSE, and INTERSECT frameworks (SLB, Houston, Texas).

The DRILLPLAN framework provides for digital well construction planning and includes features for automation of repetitive tasks and validation workflows, enabling improved quality drilling programs (e.g., digital drilling plans, etc.) to be produced quickly with assured coherency.

The PETREL framework can be part of the DELFI cognitive exploration and production (E&P) environment (SLB, Houston, Texas), referred to as the DELFI environment, for utilization in geosciences and geoengineering, for example, to analyze subsurface data from exploration to production of fluid from a reservoir.

The DELFI environment is a secure, cognitive, cloud-based collaborative environment that integrates data and workflows with digital technologies, such as artificial intelligence and machine learning. As an example, such an environment can provide for operations that involve one or more frameworks. The DELFI environment may be referred to as the DELFI framework, which may be a framework of frameworks. As an example, the DELFI framework can include various other frameworks, which can include, for example, one or more types of models (e.g., simulation models, machine learning models, etc.).

The TECHLOG framework can handle and process field and laboratory data for a variety of geologic environments (e.g., deepwater exploration, shale, etc.). The TECHLOG framework can structure wellbore data for analyses, planning, etc.

The PETROMOD framework provides petroleum systems modeling capabilities that can combine one or more of seismic, well, and geological information to model the evolution of a sedimentary basin. The PETROMOD framework can predict if, and how, a reservoir has been charged with hydrocarbons, including the source and timing of hydrocarbon generation, migration routes, quantities, and hydrocarbon type in the subsurface or at surface conditions.

The ECLIPSE framework provides a reservoir simulator (e.g., as a computational framework) with numerical solutions for fast and accurate prediction of dynamic behavior for various types of reservoirs and development schemes.

The INTERSECT framework provides a high-resolution reservoir simulator for simulation of detailed geological features and quantification of uncertainties, for example, by creating accurate production scenarios and, with the integration of precise models of the surface facilities and field operations, the INTERSECT framework can produce reliable results, which may be continuously updated by real-time data exchanges (e.g., from one or more types of data acquisition equipment in the field that can acquire data during one or more types of field operations, etc.). The INTERSECT framework can provide completion configurations for complex wells where such configurations can be built in the field, can provide detailed chemical-enhanced-oil-recovery (chemical EOR) formulations where such formulations can be implemented in the field, can analyze application of steam injection and other thermal EOR techniques for implementation in the field, advanced production controls in terms of reservoir coupling and flexible field management, and flexibility to script customized solutions for improved modeling and field management control. The INTERSECT framework, as with the other example frameworks, may be utilized as part of the DELFI environment, for example, for rapid simulation of multiple concurrent cases. For example, a workflow may utilize one or more of the DELFI environment on demand reservoir simulation features.

The aforementioned DELFI environment provides various features for workflows as to subsurface analysis, planning, construction and production, for example, as illustrated in the workspace framework 110. As shown in FIG. 1, outputs from the workspace framework 110 can be utilized for directing, controlling, etc., one or more processes in the geologic environment 150 and, feedback 160, can be received via one or more interfaces in one or more forms (e.g., acquired data as to operational conditions, equipment conditions, environment conditions, etc.).

In the example of FIG. 1, the visualization features 123 may be implemented via the workspace framework 110, for example, to perform tasks as associated with one or more of subsurface regions, planning operations, constructing wells and/or surface fluid networks, and producing from a reservoir.

As an example, a visualization process can implement one or more of various features that can be suitable for one or more web applications. For example, a template may involve use of the JAVASCRIPT object notation format (JSON) and/or one or more other languages/formats. As an example, a framework may include one or more converters. For example, consider a JSON to PYTHON converter and/or a PYTHON to JSON converter such that features can be integrated from different codes for purposes of visualization, communication, etc.

As an example, visualization features can provide for visualization of various earth models, properties, etc., in one or more dimensions. As an example, visualization features can provide for rendering of information in multiple dimensions, which may optionally include multiple resolution rendering. In such an example, information being rendered may be associated with one or more frameworks and/or one or more data stores. As an example, visualization features may include one or more control features for control of equipment, which can include, for example, field equipment that can perform one or more field operations. As an example, a workflow may utilize one or more frameworks to generate information that can be utilized to control one or more types of field equipment (e.g., drilling equipment, wireline equipment, fracturing equipment, etc.).

As an example, a model may be a simulated version of an environment, which may include one or more sites of possible emissions. As an example, a simulator may include features for simulating physical phenomena in an environment based at least in part on a model or models. A simulator, such as a weather simulator, can simulate fluid flow in an environment based at least in part on a model that can be generated via a framework that receives satellite data. A simulator can be a computerized system (e.g., a computing system) that can execute instructions using one or more processors to solve a system of equations that describe physical phenomena subject to various constraints. In such an example, the system of equations may be spatially defined (e.g., numerically discretized) according to a spatial model (e.g., of the Earth, the atmosphere, the oceans, etc.).

While several simulators are illustrated in the example of FIG. 1, one or more other simulators may be utilized, additionally or alternatively. For example, consider the VISAGE geomechanics simulator (SLB, Houston Texas) or the PIPESIM network simulator (SLB, Houston Texas), etc. The VISAGE simulator includes finite element numerical solvers that may provide simulation results such as, for example, results as to compaction and subsidence of a geologic environment, well and completion integrity in a geologic environment, cap-rock and fault-seal integrity in a geologic environment, fracture behavior in a geologic environment, thermal recovery in a geologic environment, CO2 disposal, etc. The PIPESIM simulator includes solvers that may provide simulation results such as, for example, multiphase flow results (e.g., from a reservoir to a wellhead and beyond, etc.), flowline and surface facility performance, etc. The PIPESIM simulator may be integrated, for example, with the AVOCET production operations framework (SLB, Houston Texas).

As an example, observational data such as data from one or more satellites may be utilized in the DELFI environment for one or more purposes. For example, consider integration with one or more types of frameworks. As mentioned, the VISAGE framework can perform methods concerning CO2 (e.g., CO2 disposal, etc.) and the PIPESIM framework can perform methods considering surface operations (e.g., consider gas production, separations of gas, artificial lift using gas, gas flaring, etc.).

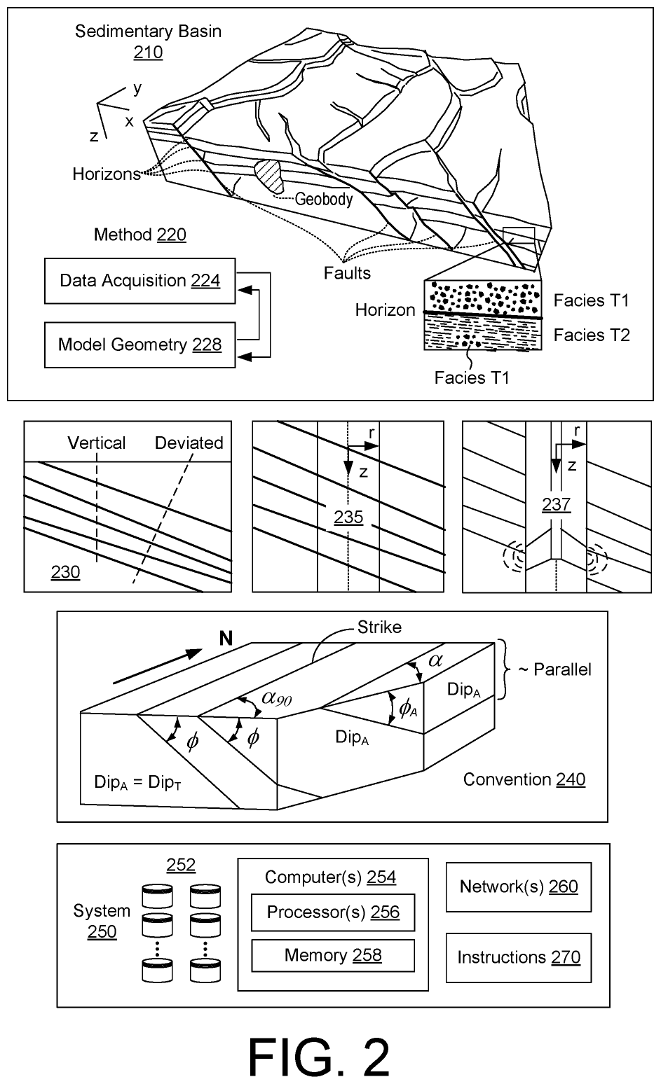

FIG. 2 shows an example of a sedimentary basin 210 (e.g., a geologic environment), an example of a method 220 for model building (e.g., for a simulator, etc.), an example of a formation 230, an example of a borehole 235 in a formation, an example of a convention 240 and an example of a system 250.

As an example, reservoir simulation, petroleum systems modeling, etc. may be applied to characterize various types of subsurface environments, including environments such as those of FIG. 1.

In FIG. 2, the sedimentary basin 210, which is a geologic environment, includes horizons, faults, one or more geobodies and facies formed over some period of geologic time. These features are distributed in two or three dimensions in space, for example, with respect to a Cartesian coordinate system (e.g., x, y and z) or other coordinate system (e.g., cylindrical, spherical, etc.). As shown, the model building method 220 includes a data acquisition block 224 and a model geometry block 228. Some data may be involved in building an initial model and, thereafter, the model may optionally be updated in response to model output, changes in time, physical phenomena, additional data, etc. As an example, data for modeling may include one or more of the following: depth or thickness maps and fault geometries and timing from seismic, remote-sensing, electromagnetic, gravity, outcrop and well log data. Furthermore, data may include depth and thickness maps stemming from facies variations (e.g., due to seismic unconformities) assumed to following geological events (“iso” times) and data may include lateral facies variations (e.g., due to lateral variation in sedimentation characteristics).

To proceed to modeling of geological processes, data may be provided, for example, data such as geochemical data (e.g., temperature, kerogen type, organic richness, etc.), timing data (e.g., from paleontology, radiometric dating, magnetic reversals, rock and fluid properties, etc.) and boundary condition data (e.g., heat-flow history, surface temperature, paleowater depth, etc.).

In basin and petroleum systems modeling, quantities such as temperature, pressure and porosity distributions within the sediments may be modeled, for example, by solving partial differential equations (PDEs) using one or more numerical techniques. Modeling may also model geometry with respect to time, for example, to account for changes stemming from geological events (e.g., deposition of material, erosion of material, shifting of material, etc.).

As shown in FIG. 2, the formation 230 includes a horizontal surface and various subsurface layers. As an example, a borehole may be vertical. As another example, a borehole may be deviated. In the example of FIG. 2, the borehole 235 may be considered a vertical borehole, for example, where the z-axis extends downwardly normal to the horizontal surface of the formation 230. As an example, a tool 237 may be positioned in a borehole, for example, to acquire information. As mentioned, a borehole tool may be configured to acquire electrical borehole images. As an example, the fullbore Formation Microlmager (FMI) tool (SLB, Houston, Texas) can acquire borehole image data. A data acquisition sequence for such a tool can include running the tool into a borehole with acquisition pads closed, opening and pressing the pads against a wall of the borehole, delivering electrical current into the material defining the borehole while translating the tool in the borehole, and sensing current remotely, which is altered by interactions with the material.

As an example, a borehole may be vertical, deviate and/or horizontal. As an example, a tool may be positioned to acquire information in a horizontal portion of a borehole. Analysis of such information may reveal vugs, dissolution planes (e.g., dissolution along bedding planes), stress-related features, dip events, etc. As an example, a tool may acquire information that may help to characterize a fractured reservoir, optionally where fractures may be natural and/or artificial (e.g., hydraulic fractures). Such information may assist with completions, stimulation treatment, etc. As an example, information acquired by a tool may be analyzed using a framework such as the TECHLOG framework (SLB, Houston, Texas).

As to the convention 240 for dip, as shown, the three dimensional orientation of a plane can be defined by its dip and strike. Dip is the angle of slope of a plane from a horizontal plane (e.g., an imaginary plane) measured in a vertical plane in a specific direction. Dip may be defined by magnitude (e.g., also known as angle or amount) and azimuth (e.g., also known as direction). As shown in the convention 240 of FIG. 2, various angles ϕ indicate angle of slope downwards, for example, from an imaginary horizontal plane (e.g., flat upper surface); whereas, dip refers to the direction towards which a dipping plane slopes (e.g., which may be given with respect to degrees, compass directions, etc.). Another feature shown in the convention of FIG. 2 is strike, which is the orientation of the line created by the intersection of a dipping plane and a horizontal plane (e.g., consider the flat upper surface as being an imaginary horizontal plane).

Some additional terms related to dip and strike may apply to an analysis, for example, depending on circumstances, orientation of collected data, etc. One term is “true dip” (see, e.g., Dip_{T} in the convention 240 of FIG. 2). True dip is the dip of a plane measured directly perpendicular to strike (see, e.g., line directed northwardly and labeled “strike” and angle α₉₀) and also the maximum possible value of dip magnitude. Another term is “apparent dip” (see, e.g., Dip_{A} in the convention 240 of FIG. 2). Apparent dip may be the dip of a plane as measured in any other direction except in the direction of true dip (see, e.g., ϕ_{A} as Dip_{A} for angle α); however, it is possible that the apparent dip is equal to the true dip (see, e.g., ϕ as Dip_{A}=Dip_{T} for angle α₉₀ with respect to the strike). In other words, where the term apparent dip is used (e.g., in a method, analysis, algorithm, etc.), fora particular dipping plane, a value for “apparent dip” may be equivalent to the true dip of that particular dipping plane.

As shown in the convention 240 of FIG. 2, the dip of a plane as seen in a cross-section perpendicular to the strike is true dip (see, e.g., the surface with ϕ as Dip_{A}=Dip_{T} for angle α₉₀ with respect to the strike). As indicated, dip observed in a cross-section in any other direction is apparent dip (see, e.g., surfaces labeled Dip_{A}). Further, as shown in the convention 240 of FIG. 2, apparent dip may be approximately 0 degrees (e.g., parallel to a horizontal surface where an edge of a cutting plane runs along a strike direction).

In terms of observing dip in wellbores, true dip is observed in wells drilled vertically. In wells drilled in any other orientation (or deviation), the dips observed are apparent dips (e.g., which are referred to by some as relative dips). In order to determine true dip values for planes observed in such boreholes, as an example, a vector computation (e.g., based on the borehole deviation) may be applied to one or more apparent dip values.

As mentioned, another term that finds use in sedimentological interpretations from borehole images is “relative dip” (e.g., Dip_{R}). A value of true dip measured from borehole images in rocks deposited in very calm environments may be subtracted (e.g., using vector-subtraction) from dips in a sand body. In such an example, the resulting dips are called relative dips and may find use in interpreting sand body orientation.

A convention such as the convention 240 may be used with respect to an analysis, an interpretation, an attribute, etc. As an example, various types of features may be described, in part, by dip (e.g., sedimentary bedding, faults and fractures, cuestas, igneous dikes and sills, metamorphic foliation, etc.). As an example, dip may change spatially as a layer approaches a geobody. For example, consider a salt body that may rise due to various forces (e.g., buoyancy, etc.). In such an example, dip may trend upward as a salt body moves upward.

Seismic interpretation may aim to identify and/or classify one or more subsurface boundaries based at least in part on one or more dip parameters (e.g., angle or magnitude, azimuth, etc.). As an example, various types of features (e.g., sedimentary bedding, faults and fractures, cuestas, igneous dikes and sills, metamorphic foliation, etc.) may be described at least in part by angle, at least in part by azimuth, etc.

As an example, equations may be provided for petroleum expulsion and migration, which may be modeled and simulated, for example, with respect to a period of time. Petroleum migration from a source material (e.g., primary migration or expulsion) may include use of a saturation model where migration-saturation values control expulsion. Determinations as to secondary migration of petroleum (e.g., oil or gas), may include using hydrodynamic potential of fluid and accounting for driving forces that promote fluid flow. Such forces can include buoyancy gradient, pore pressure gradient, and capillary pressure gradient.

As shown in FIG. 2, the system 250 includes one or more information storage devices 252, one or more computers 254, one or more networks 260 and one or more sets of instructions 270. As to the one or more computers 254, each computer may include one or more processors (e.g., or processing cores) 256 and memory 258 for storing instructions, for example, executable by at least one of the one or more processors. As an example, a computer may include one or more network interfaces (e.g., wired or wireless), one or more graphics cards, a display interface (e.g., wired or wireless), etc. As an example, imagery such as surface imagery (e.g., satellite, geological, geophysical, etc.) may be stored, processed, communicated, etc. As an example, data may include SAR data, GPS data, etc. and may be stored, for example, in one or more of the storage devices 252.

As an example, the one or more sets of instructions 270 may include instructions (e.g., stored in memory) executable by one or more processors to instruct the system 250 to perform various actions. As an example, the system 250 may be configured such that the one or more sets of instructions 270 provide for establishing one or more frameworks or a portion thereof. As an example, one or more methods, techniques, etc. may be performed using one or more sets of instructions, which may be, for example, one or more of the one or more sets of instructions 270 of FIG. 2.

As explained, phenomena associated with a sedimentary basin (e.g., a subsurface region, whether below a ground surface, water surface, etc.) may be modeled using various equations (e.g., stress, fluid flow, phase, etc.). As an example, a numerical model of a basin may find use for understanding various processes related to exploration and production of natural resources (estimating reserves in place, drilling wells, forecasting production, etc.).

For application of a numerical technique, equations may be discretized using nodes, cells, etc. For example, a numerical technique such as the finite difference method can include discretizing a 1D differential heat equation for temperature with respect to a spatial coordinate to approximate temperature derivatives (e.g., first order, second order, etc.). Where time is of interest, a derivative of temperature with respect to time may also be provided. As to the spatial coordinate, the numerical technique may rely on a spatial grid that includes various nodes where a temperature will be provided for each node upon solving the heat equation (e.g., subject to boundary conditions, generation terms, etc.). Such an example may apply to multiple dimensions in space (e.g., where discretization is applied to the multiple dimensions). Thus, a grid may discretize a volume of interest (VOI) into elementary elements (e.g., cells or grid blocks) that may be assigned or associated with properties (e.g. porosity, rock type, etc.), which may be germane to simulation of physical processes (e.g., fluid flow, reservoir compaction, etc.).

As another example of a numerical technique, consider the finite element method where space may be represented by one dimensional or multi-dimensional “elements”. For one spatial dimension, an element may be represented by two nodes positioned along a spatial coordinate. For multiple spatial dimensions, an element may include any number of nodes. Further, some equations may be represented by the total number nodes while others are represented by fewer than the total number of nodes (e.g., consider an example for the Navier-Stokes equations where fewer than the total number of nodes represent pressure). The finite element method may include providing nodes that can define triangular elements (e.g., tetrahedra in 3D, higher order simplexes in multidimensional spaces, etc.) or quadrilateral elements (e.g., hexahedra or pyramids in 3D, etc.), or polygonal elements (e.g., prisms in 3D, etc.). Such elements, as defined by corresponding nodes of a grid, may be referred to as grid cells.

Yet another example of a numerical technique is the finite volume method. For the finite volume method, values for model equation variables may be calculated at discrete places on a grid, for example, a node of the grid that includes a “finite volume” surrounding it. The finite volume method may apply the divergence theorem for evaluation of fluxes at surfaces of each finite volume such that flux entering a given finite volume equals that leaving to one or more adjacent finite volumes (e.g., to adhere to conservation laws). For the finite volume method, nodes of a grid may define grid cells.

As an example, a finite volume flow simulator may simulate phenomena using a grid where grid cells defined by the grid may include 6 faces (e.g., cuboid) addressable through three indices (e.g., such that the grid may be deemed a “structured” grid) and that geometry of the grid abides by one or more conditions (e.g., cells do not cross geologic faults and cells do not cross geologic horizons). As an example, in an effort to meet a geologic fault condition, a grid may be offset across one or more geologic faults. Construction of such a grid in a domain where topology of a fault network is complex (e.g., numerous X and Y-shaped intersections) may be non-trivial and demand resources that scale nonlinearly with increasing fault network complexity.

As an example, an approach to modeling of a sedimentary basin can include a pillar grid composed of nodes, pillars and cells. For example, in three-dimensions, eight nodes may define a cell, which may be referred to as a grid cell (e.g., a pillar grid cell). In a pillar grid model, grid cells may be indexed in an indexical domain using indexes i, j, and k (e.g., an indexical coordinate system or space, which may be represented as I, J, and K or other characters, symbols, etc.). For example, a cubic grid cell (i.e., defined by eight corner nodes) may be indexed at its shallowest lower left corner and the number of grid cells may be a product of the model's i, j and k dimensions. In such an example, each grid cell may be defined by its eight nodes, which may be labeled according to height and compass directions (e.g., basesouthwest, topsouthwest, basenorthwest, topnorthwest, etc.). Pillar grids can model, for example, faults (e.g., a surface that cuts a pillar grid), horizons (e.g., “k” index), zones (e.g., volume between two horizons), segments (e.g., contiguous block of grid cells bounded by fault planes), etc., and may be used to specify properties (e.g., earth properties).

While an indexical coordinate system is described with respect to a pillar grid, an indexical coordinate system may be used in conjunction with other types of grids. For example, a grid that can define cells may be characterized using indexes such as i, j, and k to represent three spatial dimensions. Such indexes may be capable of representing a grid, for example, in a so-called structured manner (e.g., in contrast to an unstructured manner). As to an unstructured approach, it may partition a volume of interest into different elementary volumes, for example, that may not be readily indexed following a pre-defined, repeating pattern (e.g., consider a Cartesian cube with indexes I, J, and K, along x, y, and z axes).

As an example, a structured grid may facilitate various types of operations such as those related to matrices, for example, where nearest neighbors may form clusters or bands within a matrix. In turn, a matrix may be handled using a banded solver or other suitable technique. As to a solver for an unstructured grid, as an example, it may rely on input of connectivity information that specifies how grid nodes relate to individual cells. In such an example, a matrix that may not be readily amenable to a banded or other matrix handling technique, which, in turn, can increase computational resource demands, computation time, etc.

As an example, a structured grid that includes a natural (i, j, k) indexing system can improve storage and, for example, facilitate identification of topological neighbors where cell index and connectivity might not be stored in memory and can be deduced from ordering of records/entries in memory. In such an example, storing a structured grid can use less memory than, for example, storing an unstructured grid of similar size. Further, as an example, for construction of large systems of equations (e.g., independently from their resolution), which may involve repeatedly iterating over topological neighbors of a given grid cell, such an approach may be, for example, about an order of magnitude faster when compared to use of an unstructured grid. As an example, a method that can generate a structured grid may provide compatibility with one or more frameworks (e.g., whether current, legacy, etc.).

As mentioned, where a sedimentary basin (e.g., subsurface region) includes various types of features (e.g., stratigraphic layers, faults, etc.), nodes, cells, etc., may represent, or be assigned to, such features. In turn, discretized equations may better represent the sedimentary basin and its features. As an example, a structured grid that can represent a sedimentary basin and its features, when compared to an unstructured grid, may allow for more simulations runs, more model complexity, less computational resource demands, less computation time, etc. In various examples, a structured approach and/or an unstructured approach may be utilized.

As an example, a grid may conform to structural features such as, for example, Y-faults, X-faults, low-angle unconformities, salt bodies, intrusions, etc. (e.g., geological discontinuities), to more fully capture complexity of a geological model. As an example, a grid may optionally conform to stratigraphy (e.g., in addition to one or more geological discontinuities). As to geological discontinuities, these may include model discontinuities such as one or more model boundaries. As an example, a grid may be populated with property fields generated, for example, by geostatistical methods.

As an example, a discontinuity may be discerned via seismology (e.g., seismic imaging) where, for example, a subsurface boundary or interface exists at which a physical quantity, such as the velocity of transmission of seismic waves, changes abruptly. For example, the velocity of P-waves increases from about 6.5 km/s to about 8 km/s at the Mohorovicic discontinuity between the Earth's crust and mantle.

Seismic interpretation is a process that involves examining seismic data (e.g., with respect to location and time or depth) to identify one or more types of subsurface structures (e.g., horizons, faults, geobodies, etc.). An interpretation process may consider vertical seismic sections, inline and crossline directions, horizontal seismic sections called horizontal time slices, etc. Seismic data may optionally be interpreted with other data such as, for example, well log data.

As an example, seismic interpretation may be performed using seismic to simulation software such as the PETREL seismic to simulation software framework (SLB, Houston, Texas), which includes various features, for example, to perform attribute analyses (e.g., with respect to a 3D seismic cube, a 2D seismic line, etc.), to analyze other data, to build models, etc. While the PETREL seismic to simulation software framework is mentioned, other types of software, frameworks, etc., may be employed.

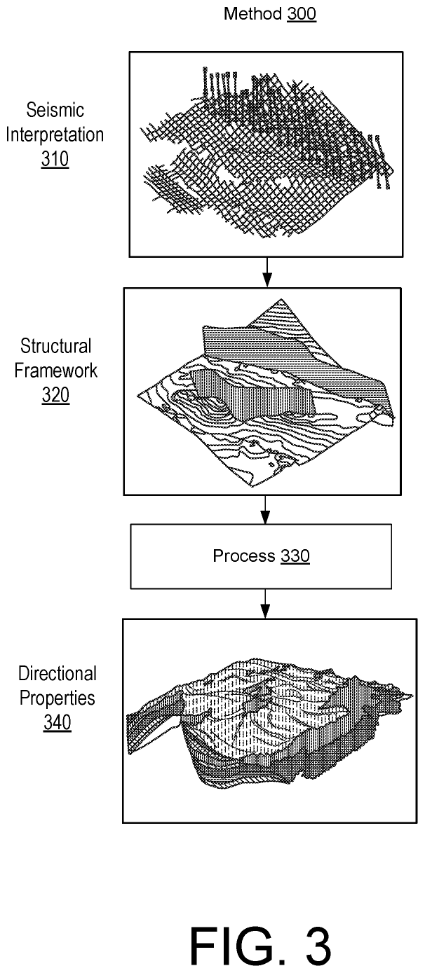

FIG. 3 shows an example of a method 300 that includes a seismic interpretation block 310 for accessing at least seismic data as acquired by one or more sensors that sense seismic energy that travels in a subterranean environment, for performing a seismic interpretation based at least in part on at least a portion of the data and generating information (e.g., locations of structures, etc.). The method 300 also includes a structural framework block 320 for generating one or more structures (e.g., structures within a model, etc.) that represent physical structures in the subterranean environment. For example, interpretation may identify a horizon and structural model can include that horizon as a data-based structure with a likelihood that it actually exists in the subterranean environment given variations in seismic data (e.g., variations in traces, etc.). The method 300 includes a processing block 330, which may implement one or more techniques that can enhance how properties are handled where such properties can include one or more directional properties, which can be assigned to portions of a grid per the property block 340.

As an example, the directional properties as in the property block 340 can be stored in association with the grid, for example, in a storage device. Such a storage device can be part of a reservoir simulator or otherwise accessible by a reservoir simulator via one or more interfaces. For example, consider a reservoir simulator that includes one or more processors and memory where data and/or instructions stored in a storage device can be loaded into the memory via operation of at least one of the one or more processors.

As explained, a simulation run may be lengthy, depending on the number of equations (e.g., as may be associated with grid size, etc.). As an example, a simulation run may be of the order of hours or, for example, days. As may be appreciated, computer resources tied up for hours executing a simulation run may, where stability is lacking, not generate a result or not generate a meaningful result.

A lower run turnaround time can increase benefits from a simulation study allotted a budgeted time period. As a corollary, time spent in repeated runs fighting model instabilities or time-stepping can be counterproductive. Various factors can affect run time, for example, run time can equal the product (CPU/GPU time/step)×(number of timesteps). The first factor tends to be large and the second factor tends to be small for an implicit formulation, and conversely for the IMPES formulation. IMPES is a conditionally stable formulation that involves a timestep Δt<Δt* to reduce risk of oscillations and error growth, where Δt* can be defined as a maximum stable timestep. For IMPES, the conditional stability stems from the explicit treatment of nonpressure variables in interblock flow terms.

As explained, a simulator may utilize a preconditioner, for example, consider Nested Factorization (NF) and incomplete LU factorization (ILU(n)). The term “LU factorization” refers to the factoring of a matrix A into the product of a lower triangular matrix L and an upper triangular matrix U, which can be computationally expensive and involve Gaussian elimination. The term “ILU(n)” denotes incomplete LU factorization, where limited fill-in is allowed and n is the order of fill.

NF can perform suitably under particular conditions such as when transmissibilities associated with a particular direction in a grid dominate those in other directions uniformly throughout the grid. ILU(n) or red-black ILU(n) tend to be less sensitive than NF to ordering of blocks and spatial variation of direction of dominant transmissibilities.

As explained, transmissibility can be a measure of conductivity of a formation as to fluid where transmissibility may be defined by a particular simulator in a particular manner.

As to the INTERSECT simulator, it uses a computational solver that can implement preconditioning where such preconditioning can involve algebraically decomposing a system of equations into subsystems that may be handled based on their particular characteristics to facilitate solution. Resulting reservoir equations can be solved numerically by iterative techniques until convergence is reached for the entire system of equations, which can account for one or more wells, one or more surface facilities, etc.

The INTERSECT simulator framework can provide outputs for field tasks as to field operations, which may include operations as to surface facilities. As to an example of a workflow, consider a simulation that accounts for over 100 producing well and includes millions of cells in a grid, which can structurally model more than 10,000 fractures. As an example, time during execution of a run can include tasks such as evaluate residual and assemble Jacobian matrix, linear solver, nonlinear update and nonlinear convergence test. In such an example, time may be predominantly spent on the linear solver, followed by the evaluation of residual and assembly of the Jacobian matrix (e.g., or portions thereof, etc.).

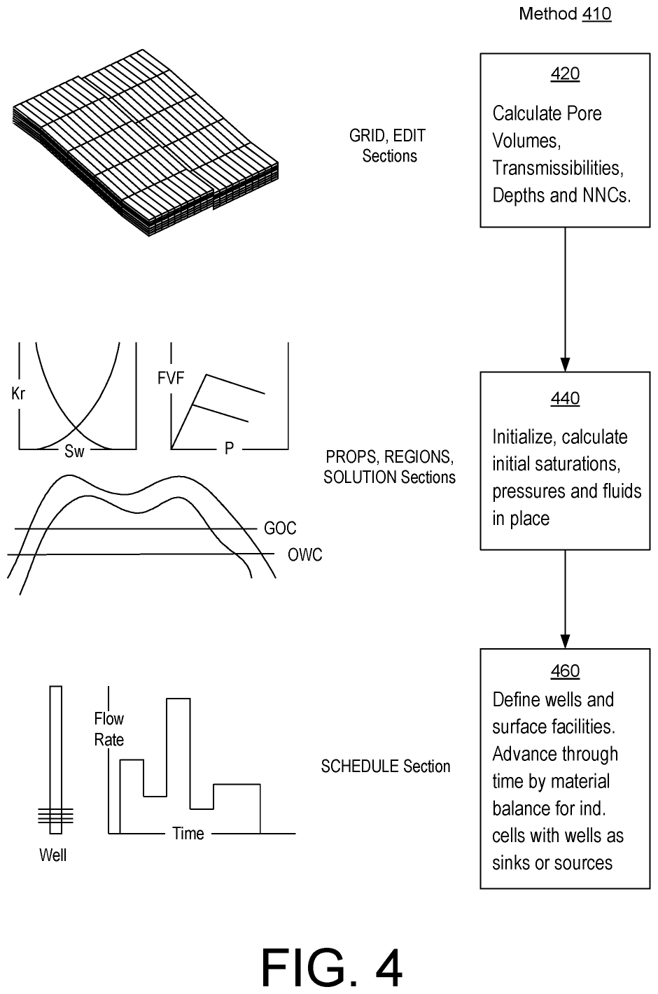

FIG. 4 shows an example of a method 410 that includes a calculation block 420 for calculating pore volumes, transmissibilities, depths and non-neighbor connections (NNCs), an initialization and calculation block 440 for initializing and calculating initial saturations, pressure and fluids in place, and a definition and time progression block 460 for defining one or more wells and surface facilities and advancing through time, for example, via material balances for individual cells (e.g., with the one or more wells as individual sinks and/or sources). In such an example, the block 420 may include one or more sets of instructions for generating, adjusting, etc. one or more grids, which may represent one or more faults, a portion of a fault, etc.

As an example, a method can include operating a reservoir simulator to generate simulation results and determining a well trajectory using the generated simulation results and/or constructing a well via drilling into a reservoir using the generated simulation results. As explained, a method can improve reservoir simulator operation to generate more accurate simulation results that improve one or more field operations associated with production of fluid from a reservoir. Such a method improves operation of a reservoir simulator that performs a technological process in the realm of physical objects and physical substances such as a reservoir being a physical object and fluid being a physical substance. As an example, a workflow can include seismic imaging to generate seismic images, identifying structures associated with a reservoir using the seismic images, generating a model that includes the reservoir, assigning directional properties to the model, operating a reservoir simulator to generate fluid flow patterns using the model and the directional properties, drilling a borehole according to a trajectory determined using the fluid flow patterns, completing a well using the borehole, and producing fluid from the reservoir using the well. As an example, drilling a borehole according to a trajectory determined using the fluid flow patterns may also include using directional permeabilities. For example, directional permeabilities may provide an indication of formation properties associated with stability of a borehole in a formation. As an example, an angle of a borehole may be determined using directional permeabilities as the angle may provide for increases stability while maintaining the borehole in a desired reservoir region where a completed well can include perforations that may be directional where direction is based at least in part on directional permeability. As an example, a hydraulic fracturing workflow can include well placement and/or fracture placement based at least in part on directional permeabilities, as may be taken into account by fluid flow simulation results and/or as may be taken into account by hydraulic fracturing simulation results (e.g., by a hydraulic fracturing simulator). In such an example, fractures may be generated using an equipment configuration that aims to generate fractures that provide for favorable drainage of fluid from a reservoir (e.g., fracture orientation with respect to directional permeability). As an example, fractures tend to grow in a particular orientation with respect to a permeability tensor orientation. As an example, a reservoir simulator may simulate a hydraulically fractured reservoir using directional permeabilities that account for one or more hydraulic fractures.

As an example, a method can include utilizing permeability where permeability defines an ability to allow flow to occur at one or more points. As an example, a method can include utilizing transmissibility where transmissibility defines an ability to allow flow between points. As an example, transmissibility can be defined as a measure of conductivity of rock (e.g., reservoir rock, etc.) as may be adjusted by viscosity of flowable fluid (e.g., reservoir fluid, injected fluid, etc.). As an example, a simulator can include computing transmissibility between various cell centers of cells defined by a grid.

As to the initialization and calculation block 440, for an initial time (e.g., to), saturation distribution within a grid model of a geologic environment and pressure distribution within the grid model of the geologic environment may be set to represent an equilibrium state (e.g., a static state or “no-flow” state), for example, with respect to gravity. As an example, to approximate the equilibrium state, calculations can be performed. As an example, such calculations may be performed by one or more sets of instructions. For example, one or more of a seismic-to-simulation framework, a reservoir simulator, a specialized set of instructions, etc. may be implemented to perform one or more calculations that may aim to approximate or to facilitate approximation of an equilibrium state. As an example, a reservoir simulator may include a set of instructions for initialization using data to compute capillary and fluid gradients, and hence fluid saturation densities in individual cells of a grid model that represents a geologic environment.

As an example, a reservoir simulator may advance in time. As an example, a numeric solver may be implemented that can generate a solution for individual time increments (e.g., points in time). As an example, a solver may implement an implicit solution scheme and/or an explicit solution scheme, noting that an implicit solution scheme may allow for larger time increments than an explicit scheme. Times at which a solution is desired may be set forth in a “schedule”. For example, a schedule may include smaller time increments for an earlier period of time followed by larger time increments.

A solver may implement one or more techniques to help assure stability, convergence, accuracy, etc. For example, when advancing a solution in time, a solver may implement sub-increments of time, however, an increase in the number of increments can increase computation time. As an example, an adjustable increment size may be used, for example, based on information of one or more previous increments.

As an example, a numeric solver may implement one or more of a finite difference approach, a finite element approach, a finite volume approach, a meshless approach, etc. As an example, the ECLIPSE reservoir simulator can implement central differences for spatial approximation and forward differences in time. As an example, a matrix that represents grid cells and associated equations may be sparse, diagonally banded and blocked as well as include off-diagonal entries. As an example, the INTERSECT simulator may be implemented.

As an example, a solver may implement an implicit pressure, explicit saturation (IMPES) scheme. Such a scheme may be considered to be an intermediate form of explicit and implicit techniques. In an IMPES scheme, saturations are updated explicitly while pressure is solved implicitly. As an example, a solver may implement an implicit scheme that may be adaptive (e.g., an adaptive implicit scheme).

As to conservation of mass, values (e.g., for water, gas and oil) in individual cells of a grid cell model may be specified to sum to a certain value, which may be considered a control criterion for mass conservation. As black oil equations tend to be non-linear, a Newton-Raphson type of technique may be implemented, which includes determining derivatives, iterations, etc. For example, a solution may be found by iterating according to the Newton-Raphson scheme where such iterations may be referred to as non-linear iterations, Newton iterations or outer iterations. Where one or more error criteria are fulfilled, the solution procedure has converged, and a converged solution has been found. Thus, within a Newton iteration, a linear problem is solved by performing a number of linear iterations, which may be referred to as inner iterations.

As an example, a solution scheme may be represented by the following pseudo-algorithm:

```
// Pseudo-algorithm for Newton-Raphson for systems
initialize(v);
do {
  //Non-linear iterations
  formulate_non_linear_system(v);
  make_total_differential(v);
  do {
    // Linear iterations:
    update_linear_system_variables(v);
  }
  while((linear_system_has_not_converged(v));
  update_non_linear_system_after_linear_convergence(v);
}
while((non_linear_system_has_not_converged(v))
```

As an example, a solver may perform a number of inner iterations (e.g., linear) and a number of outer iterations (e.g., non-linear). As an example, a number of inner iterations may be of the order of about 10 to about 20 within an outer iteration while a number of outer iterations may be about ten or less for an individual time increment.

As mentioned, a grid may be revised (e.g., adjusted, etc.) based at least in part on simulation results, which may optionally include results such as convergence behavior. For example, where convergence may be possibly improved, one or more adjustments may be made to a grid where such one or more adjustments may allow for convergence, expedite convergence, etc.

As explained, a system can provide for decision making that can improve operation a simulator for performing simulation tasks that aim to generate results as to one or more physical phenomena. While various examples refer to a reservoir simulator, one or more other types of simulators may be utilized with such a system.

As an example, a system can provide for implementing a series of grid reorderings (referred to as “colorings”) for a simulation grid such that cells are grouped so that there exist groups of continuous cells with no adjacency, or with adjacency which will be automatically disregarded by a preconditioner within this reordering. While grid and cells are mentioned, as explained, such an approach may be applied to various types of discretizations, including, for example, meshless approaches to discretization.

In various examples, groups can be referred to as “colors” or color groups. The notion of colors stems from color theory, or the color theorem. For example, the four color theorem, or the four color map theorem, states that no more than four colors are required to color the regions of a map so that no two adjacent regions have the same color. In such a context, adjacent means that two regions share a common boundary curve segment, not merely a corner where three or more regions meet. While “color” and “colors” are mentioned, other indicators may be utilized for distinguishing regions.

As an example, a system can provide for selection amongst a group coloring schemes where each coloring scheme uses a separate definition of which adjacencies may be disregarded. For example, such a system can select colors which avoid the dropping or reordering of strong connections that will reduce the quality of a preconditioner. Such an approach allows both properties of a preconditioner and parallel efficiency to be controlled by a coloring scheme, optionally without modification of a preconditioner itself.

As to some examples of schemes, consider “General”, “Strong”, “Vertical_Strong” and “Vertical_Weak”; noting that directionality may differ depending on model domain. For example, consider utilization of directionality that may differ from vertical and/or horizontal. As explained, in various reservoir simulation models, discretization can differ between vertical and horizontal dimensions such that one or more aspect ratios may be defined. As mentioned, various characteristics of a reservoir can be differ spatially and various characteristics can depend on geometry once included in a model (e.g., consider transmissibility).

As to General, it does not disregard connections and orders colors using an oscillating pattern, first cycling forwards through the colors, then cycling backwards.

As to Strong, it considers only connections above an arbitrary threshold of the maximum for each cell, and colors the grid according to this reduced set of connections. In Strong, weak connections which have cells of the same color adjacent to each other are then disregarded.

As to Vertical_Strong, it identifies vertical columns and colors these independently. In Vertical_Strong, areal connections are then disregarded where colors either side of a connection match.

As to Vertical_Weak, it identifies vertical columns and colors these independently. In Vertical_Weak, the top cell in each column is then assigned a new color which is consistent with its horizontal neighbors and the rest of the column's colors are modified similarly to maintain a consistent coloring within the column.

As an example, a method can include using a set of example simulation models and comparing the performance of simulation using the different coloring schemes within the colored preconditioning scheme such as MC-DILU, MC-ILU or some other colored preconditioning scheme. As an example, a system can allow for the use of different numbers of colors within each coloring scheme. As to the aforementioned method, it can include analyzing the models' performance according to grid features, such as the structure of the grid, the presence of local grid refinements and the ratio of horizontal to vertical permeability or transmissibility (hereafter referred to as “anisotropy ratio”). Such a method can further include constructing a machine model (e.g., a decision tree, etc.) based upon these features utilizing one or more threshold values for permeability or transmissibility anisotropy ratio.

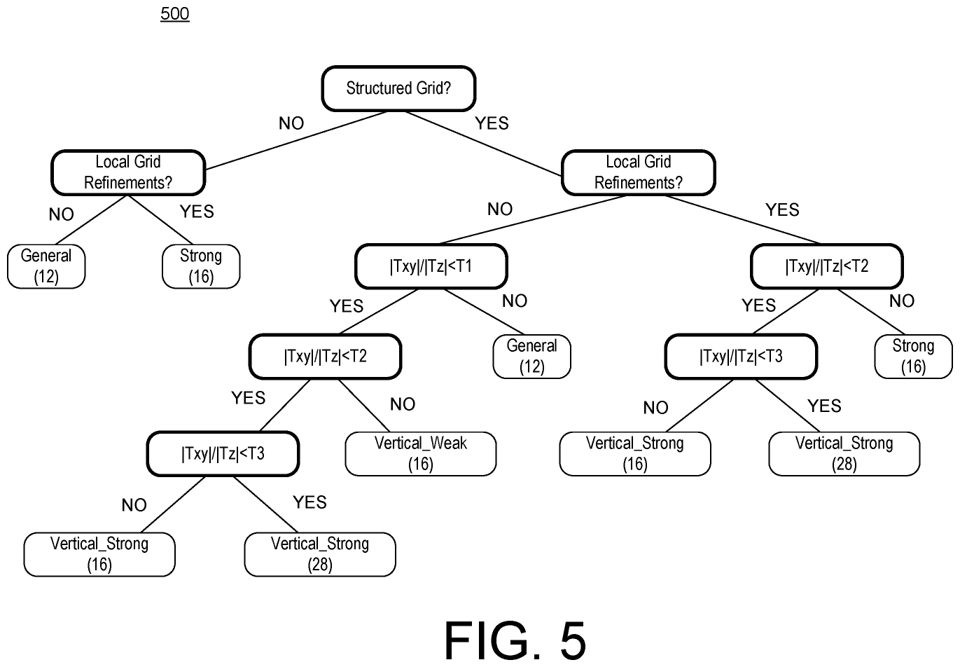

FIG. 5 shows an example of a machine model 500 that can be stored in memory of a memory device accessible to one or more processors (e.g., cores, CPUs, GPUs, etc.). In the example of FIG. 5, the machine model 500 is shown including a decision tree. As explained, such a machine model 500 can be generated using data, which can include simulation data from execution of one or more simulators. As explained, a machine model can provide for decision making to improve a simulation workflow, including operation of a simulator. As explained, examples of schemes within a group of schemes can include one or more of General, Strong, Vertical_Strong and Vertical_Weak. These schemes are shown as examples in the machine model 500 of FIG. 5 along with numbers in parentheses to indicate the number of colors utilized. As explained, the terms color and colors are utilized to comport with map theory and various so-called multicolor preconditioners; noting that other indicators may be utilized as a substitute for color.

As an example, a system can include features that implement executable code to determine an anisotropy ratio and one or more other machine model (e.g., decision tree, etc.) characteristics. For example, at the start of a simulation (and, if chosen, thereafter at routine intervals where a decision tree characteristic is dynamic), a system can provide for evaluation of the best coloring scheme using the machine model (e.g., decision tree, etc.) and utilization of the coloring scheme determined.

As an example, a system can provide for utilizing a coloring scheme selected (e.g., suggested), for example, with a corresponding number of colors (e.g., determined, suggested, etc.) as a first attempt. In such an example, one or more additional colors may be demanded because, in various cases, it may not be possible to reorder cells such that the conditions of the coloring scheme are met with the number of colors selected. As an example, one or more of certain coloring schemes may disregard connections rather than recoloring.

As explained with respect to the machine model 500 of FIG. 5, an example of a decision tree machine model is shown where a selected coloring scheme and number of colors (indicated in parentheses) depends on properties of a simulation model, such as, for example, transmissibility anisotropy of a reservoir simulation model.

In the example of FIG. 5, the machine model 500 includes decision blocks that can be utilized to progress to various branches and ultimately to one of the leaves. As shown, at the top, the machine model 500 includes a decision block as to “structured grid”. If a structured grid is indicated or detected, the machine model 500 progress via a “yes” branch to a local grid refinements decision block for structured grids; whereas, if a structured grid is not indicated or detected (e.g., an unstructured grid is indicated or detected), the machine model 500 progresses to a local grid refinements decision block for unstructured grids, which branches to two leaves, one for General with 12 colors and the other for Strong with 16 colors.

As to the branches stemming from the local grid refinement decision block for structured grids, they further branch to decision blocks that can compare a ratio to a respective threshold. For example, T can represent transmissibility where Txy is areal plane transmissibility (e.g., lateral) and where Tz is vertical transmissibility (e.g., depth). As shown, a ratio can be computed using the absolute value of Txy divided by the absolute value of Tz, or vice versa. In the example of FIG. 5, three threshold values are shown: T1, T2 and T3, where T1 is greater than T2 and T2 is greater than T3. As shown, the selectable options, represented as end points of the machine model 500 (e.g., leaves of a decision tree), can be tiered, for example, from General to Strong, to Vertical_Weak, to Vertical_Strong, where number of colors may be increased (e.g., from 16 to 28, etc.), as appropriate.

As an example, a system can include features for incorporating a measure of the disregarded fill terms into a machine model (e.g., a decision tree, etc.). For example, by calculating an approximate error associated with different coloring options, a machine model can be refined to better handle a wider range of situations.

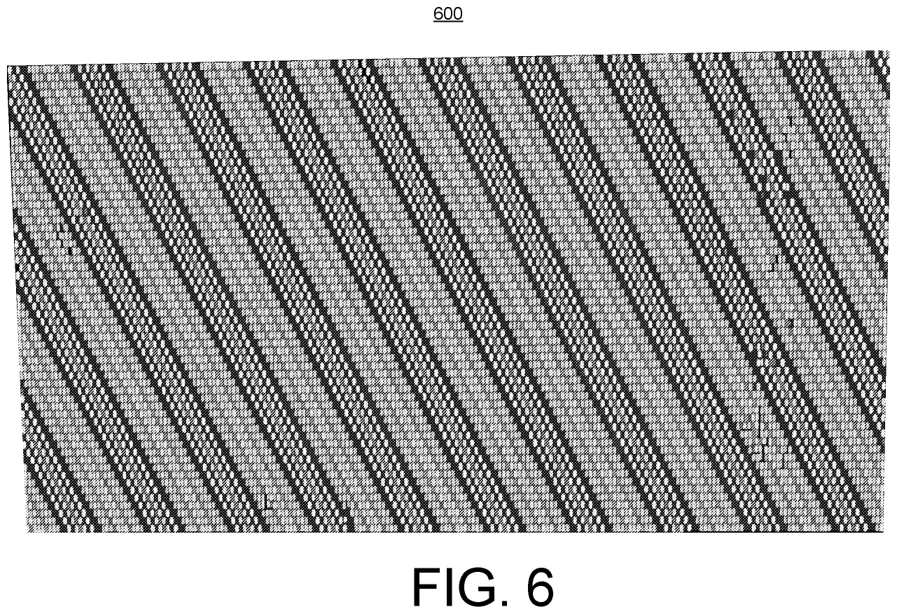

FIG. 6 shows an example of a portion of a simulation model 600 (e.g., as a spatial model and/or as a data structure) that is shown in grayscale where different shades of gray represent colors according to a coloring scheme selected by a machine model (e.g., a trained machine model).

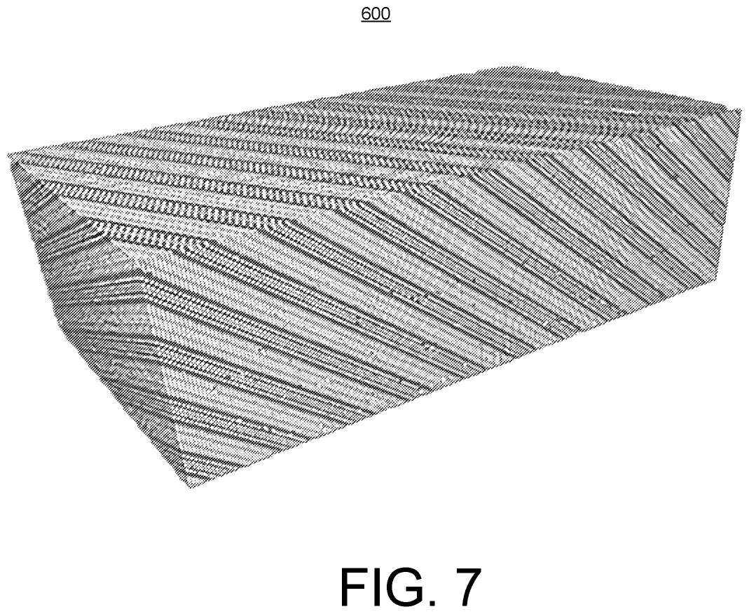

FIG. 7 shows a perspective view of the simulation model 600 (e.g., as a spatial model and/or as a data structure) that is shown in grayscale where different shades of gray represent colors according to a coloring scheme selected by a machine model (e.g., a trained machine model).

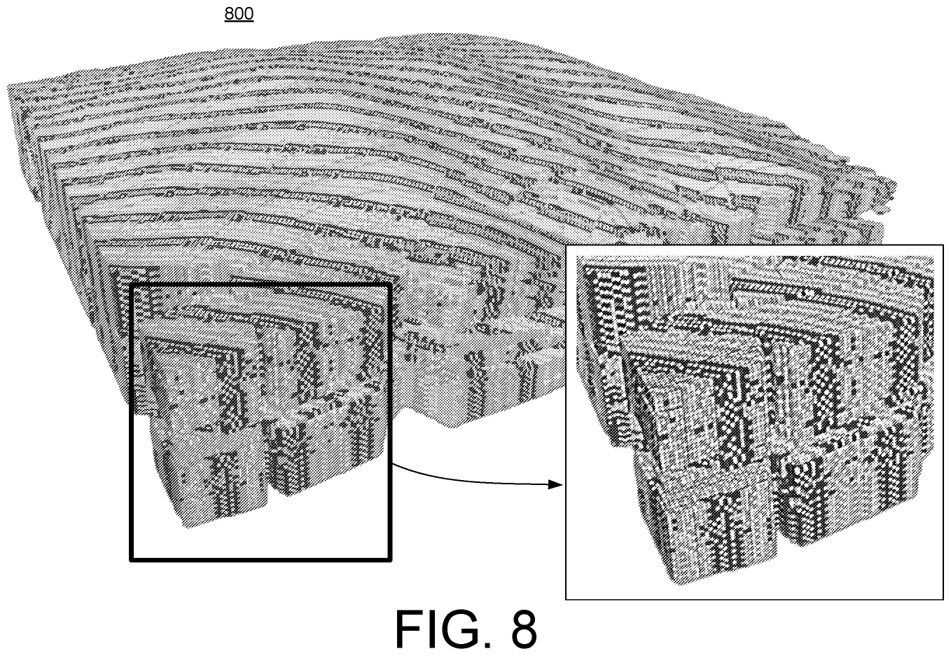

FIG. 8 shows a perspective view of an example of a simulation model 800 (e.g., as a spatial model and/or as a data structure) that is shown in grayscale where different shades of gray represent colors according to a coloring scheme selected by a machine model (e.g., a trained machine model).

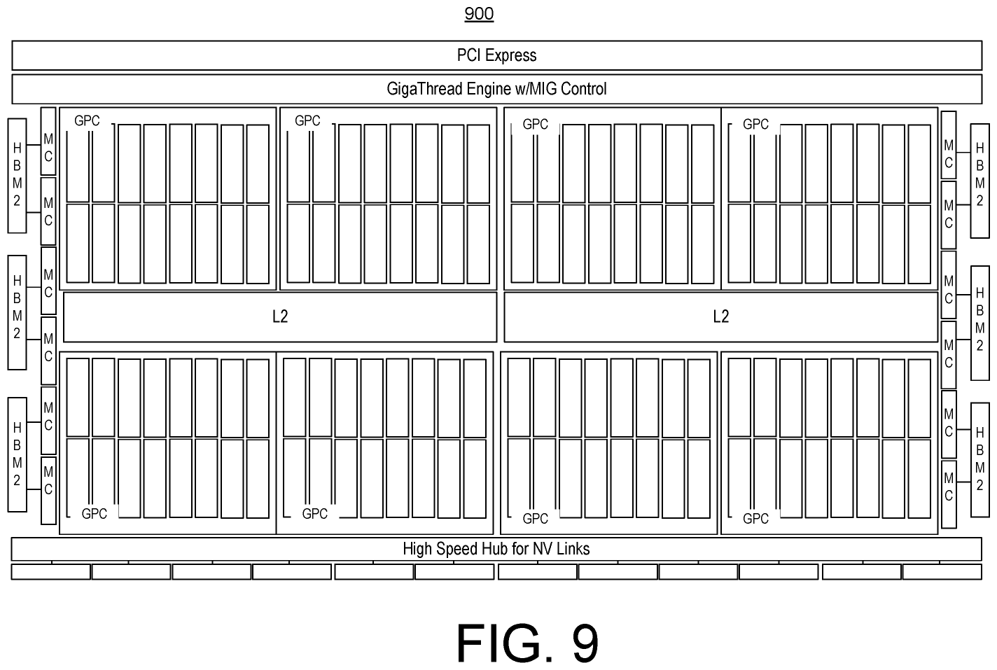

FIG. 9 shows an example of a GPU hardware architecture 900. In the example of FIG. 9, the GPU hardware architecture corresponds to a NVIDIA A100 GPU hardware architecture (Nvidia Corporation, Santa Clara, CA) and shows a full GA100 GPU with 128 streaming multiprocessors (SMs); noting that the A100 is based on GA100 and has 108 SMs. The NVIDIA GA100 GPU is composed of multiple GPU processing clusters (GPCs), texture processing clusters (TPCs), SMs, and high-bandwidth memory 2 (HBM2) memory controllers. The full implementation of the GA100 GPU includes the following units: 8 GPCs, 8 TPCs/GPC, 2 SMs/TPC, 16 SMs/GPC, 128 SMs per full GPU; 64 FP32 CUDA Cores/SM, 8192 FP32 CUDA Cores per full GPU; 4 third-generation Tensor Cores/SM, 512 third-generation Tensor Cores per full GPU; and 6 HBM2 stacks, 12 512-bit memory controllers.

The A100 Tensor Core GPU implementation of the GA100 GPU includes the following units: 7 GPCs, 7 or 8 TPCs/GPC, 2 SMs/TPC, up to 16 SMs/GPC, 108 SMs; 64 FP32 CUDA Cores/SM, 6912 FP32 CUDA Cores per GPU; 4 third-generation Tensor Cores/SM, 432 third-generation Tensor Cores per GPU; and 5 HBM2 stacks, 10 512-bit memory controllers. The A100 Tensor Core GPU can include 40 GB of high-speed HBM2 memory with 1555 GB/sec of memory bandwidth and can include a 40 MB Level 2 (L2) cache with a partitioned crossbar structure.

As an example, a simulator can include or be operatively coupled to multiple processors, cores, etc. For example, consider implementation of a linear solver that can access multiple processors, cores, etc. In such an example, the simulator can utilize large-scale parallel capability of hardware such as one or more GPUs. As an example, computation of properties and the linearization of reservoir equations can take place on a GPU, making them faster than a single CPU implementation. As an example, a simulator can include reporting code that aims to increase performance, for example, when using one or more GPUs for performing simulation tasks.

As an example of a reservoir simulation, consider a waterflood scenario in a reservoir with a fluvial channel that includes 2.3 million active cells, where a challenge exists due to a high petrophysical contrast between a main channel and background facies, and a permeabilities range from 1 to 1,500 mD. In such an example, a field management approach can indicate a drilling pattern of 16 producers and 14 water injectors for sequential drilling as rigs become available. In such an example, a reservoir simulator can generate results that can be utilized to estimate a field life (e.g., viable production of hydrocarbons). For example, consider prediction of a field life of 58 years.

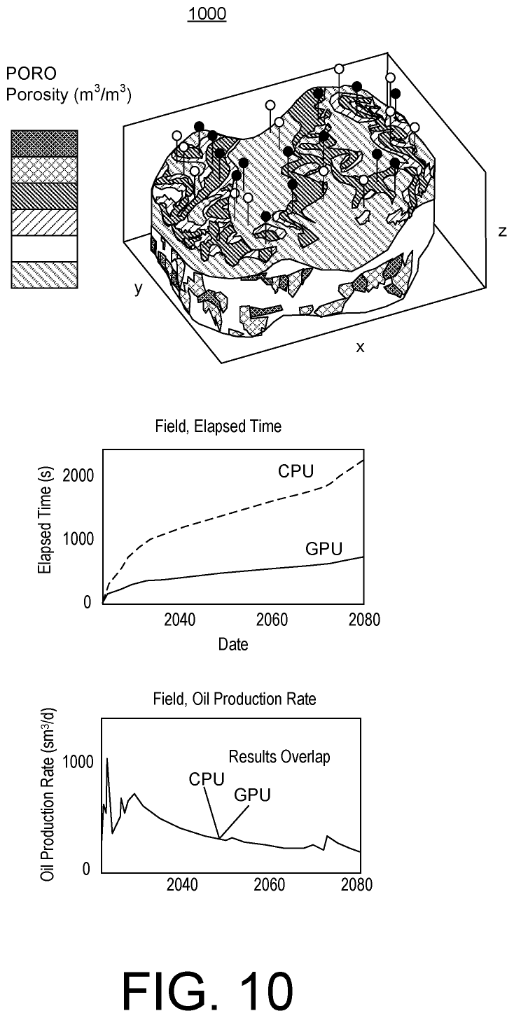

In the aforementioned example, trials compared the use of a single computing node, where 48 CPU cores are available (2 AMD 7352, Advanced Micro Devices, Inc., Santa Clara, CA) with an NVIDIA A100 GPU card. Running this model in the INTERSECT simulator in both CPU and GPU formulations provided same or similar results, without changes to the data deck (see, e.g., FIG. 10). In the trails, the GPU implementation outperformed the 48-way CPU run by a factor of three (see, e.g., FIG. 10). Thus, the GPU implementation improved simulator performance.

FIG. 10 shows example plots 1000 of porosity for the aforementioned waterflood scenario model, elapsed time versus date (e.g., from 2020 to 2080) for the CPU and GPU approaches, and results of the two approaches for oil production rate with respect to time (e.g., from 2020 to 2080). As shown, the GPU approach can improve performance of a simulator compared to the CPU approach in that elapsed time is less for the GPU approach while results from each of the approaches overlap. As an example, simulation results can be utilized for decision making as to field operations and/or control of field operations where, for example, more timely results can provide for more timely decision making. Such results may also be utilized for model refinements, which may be utilized to generate a refined model. In such an example, a refined model may be developed more rapidly through use of a GPU approach. As shown, for porosity, it can be a property that various within a geologic region. Porosity variations can stem from geologic processes that have occurred over many years and, for example, a region of interest with favorable porosity may be subjected to refinement (e.g., local grid refinement). As to phenomena that can contribute to variations in porosity, consider channeling where a channel can form as a commonly concave-based depression through which water and sediment flow and into which sediment can be deposited in distinctive, often elongated bodies. Channels can occur in a variety of morphologies (e.g., straight, meandering or braided). In some areas, coarse sediments can fill channels of streams or rivers that cut through finer grained sediments or rocks. The close proximity of coarse-grained and fine-grained sediments can ultimately lead to the formation of stratigraphic hydrocarbon traps.

As an example, a system may be utilized to facilitate parallel computing for a simulator where a simulation model is analyzed by a machine model or machine models for anisotropy with respect to discretization (e.g., grid, etc.). As explained, for reservoir simulation, transmissibility depends on discretization geometry such that transmissibility can be used as input to a machine model that can make decisions as to one or more approaches to preconditioning.

As explained, a machine model can be or include a decision tree, which can be implemented as a decision support tool that uses a tree-like model of decisions and their possible consequences, which may include chance event outcomes, resource costs, and utility. A decision tree can include conditional control statements.

As to decision tree learning or induction of decision trees, it can be implemented as a predictive modeling approach. For example, consider learning using one or more of statistics, data mining and machine learning. As an example, a decision tree can be a predictive model to go from observations about an item (e.g., represented in branches) to conclusions about the item's target value (e.g., represented in the leaves). Hence, a decision tree can include one or more inputs and one or more outputs. With reference to the example of machine model 500 of FIG. 5, observations can include one or more of structure/unstructured, refined/unrefined, transmissibility ratio, etc., where target values can include one or more color schemes.

As an example, a machine model can be a classification model. For example, leaves of a decision tree can represent class labels and branches represent conjunctions of features that lead to those class labels. In decision analysis, a decision tree can be used to visually and explicitly represent decisions and decision making. As an example, a system can provide for generating information that can be rendered to a display such that a user (e.g., a simulationist) can observe operation of a machine model to understand what technique or techniques are being applied or to be applied in simulation by a simulator.

Some techniques, sometimes referred to as ensemble methods, can involve constructing more than one machine model. As an example, consider an ensemble method that constructs multiple decision trees.

As an example, a boosted tree approach can incrementally build an ensemble by training each new instance to emphasize the training instances previously mis-modeled. For example, consider the AdaBoost technique. Boosted trees can be used for regression-type and classification-type problems.

As to a bootstrap aggregated (or bagged) decision tree approach, it can include building multiple decision trees by repeatedly resampling training data with replacement, and voting the trees for a consensus prediction. As an example, a random forest classifier can be a specific type of bootstrap aggregating

As to a rotation forest, it can involve training individual decision trees by first applying principal component analysis (PCA) on a random subset of the input features.

Some examples of decision tree techniques include ID3 (Iterative Dichotomiser 3); C4.5 (successor of ID3); CART (Classification And Regression Tree); Chi-square automatic interaction detection (CHAID); MARS; and Conditional Inference Trees (e.g., a statistics-based approach that uses non-parametric tests as splitting criteria, corrected for multiple testing to avoid overfitting, which can result in unbiased predictor selection and does not require pruning).

As an example, a machine model can leverage concepts of fuzzy set theory for as in Fuzzy Decision Tree (FDT). In this type of fuzzy classification, an input vector can be associated with multiple classes, each with a different confidence value. As an example, boosted ensembles of FDTs may be utilized.

As to types of machine learning models, consider one or more of a support vector machine (SVM) model, a k-nearest neighbors (KNN) model, an ensemble classifier model, a neural network (NN) model, etc. As an example, a machine learning model can be a deep learning model (e.g., deep Boltzmann machine, deep belief network, convolutional neural network, stacked auto-encoder, etc.), an ensemble model (e.g., random forest, gradient boosting machine, bootstrapped aggregation, AdaBoost, stacked generalization, gradient boosted regression tree, etc.), a neural network model (e.g., radial basis function network, perceptron, back-propagation, Hopfield network, etc.), a regularization model (e.g., ridge regression, least absolute shrinkage and selection operator, elastic net, least angle regression), a rule system model (e.g., cubist, one rule, zero rule, repeated incremental pruning to produce error reduction), a regression model (e.g., linear regression, ordinary least squares regression, stepwise regression, multivariate adaptive regression splines, locally estimated scatterplot smoothing, logistic regression, etc.), a Bayesian model (e.g., naïve Bayes, average on-dependence estimators, Bayesian belief network, Gaussian naïve Bayes, multinomial naïve Bayes, Bayesian network), a decision tree model (e.g., classification and regression tree, iterative dichotomiser 3, C4.5, C5.0, chi-squared automatic interaction detection, decision stump, conditional decision tree, M5), a dimensionality reduction model (e.g., principal component analysis, partial least squares regression, Sammon mapping, multidimensional scaling, projection pursuit, principal component regression, partial least squares discriminant analysis, mixture discriminant analysis, quadratic discriminant analysis, regularized discriminant analysis, flexible discriminant analysis, linear discriminant analysis, etc.), an instance model (e.g., k-nearest neighbor, learning vector quantization, self-organizing map, locally weighted learning, etc.), a clustering model (e.g., k-means, k-medians, expectation maximization, hierarchical clustering, etc.), etc.

As an example, the TENSORFLOW framework (Google LLC, Mountain View, California) may be implemented, which is an open source software library for dataflow programming that includes a symbolic math library, which can be implemented for machine learning applications that can include neural networks. As an example, the CAFFE framework may be implemented, which is a DL framework developed by Berkeley AI Research (BAIR) (University of California, Berkeley, California). As another example, consider the SCIKIT platform (e.g., scikit-learn), which utilizes the PYTHON programming language. As an example, a framework such as the APOLLO AI framework may be utilized (APOLLO.AI GmbH, Germany). As mentioned, a framework such as the PYTORCH framework may be utilized.

As an example, a training method can include various actions that can operate on a dataset to train a ML model. As an example, a dataset can be split into training data and test data where test data can provide for evaluation. A method can include cross-validation of parameters and best parameters, which can be provided for model training.

The TENSORFLOW framework can run on multiple CPUs and GPUs (with optional CUDA (NVIDIA Corp., Santa Clara, California) and SYCL (The Khronos Group Inc., Beaverton, Oregon) extensions for general-purpose computing on graphics processing units (GPUs)). TENSORFLOW is available on 64-bit LINUX, MACOS (Apple Inc., Cupertino, California), WINDOWS (Microsoft Corp., Redmond, Washington), and mobile computing platforms including ANDROID (Google LLC, Mountain View, California) and IOS (Apple Inc.) operating system based platforms.

TENSORFLOW computations can be expressed as stateful dataflow graphs; noting that the name TENSORFLOW derives from the operations that such neural networks perform on multidimensional data arrays. Such arrays can be referred to as “tensors”.

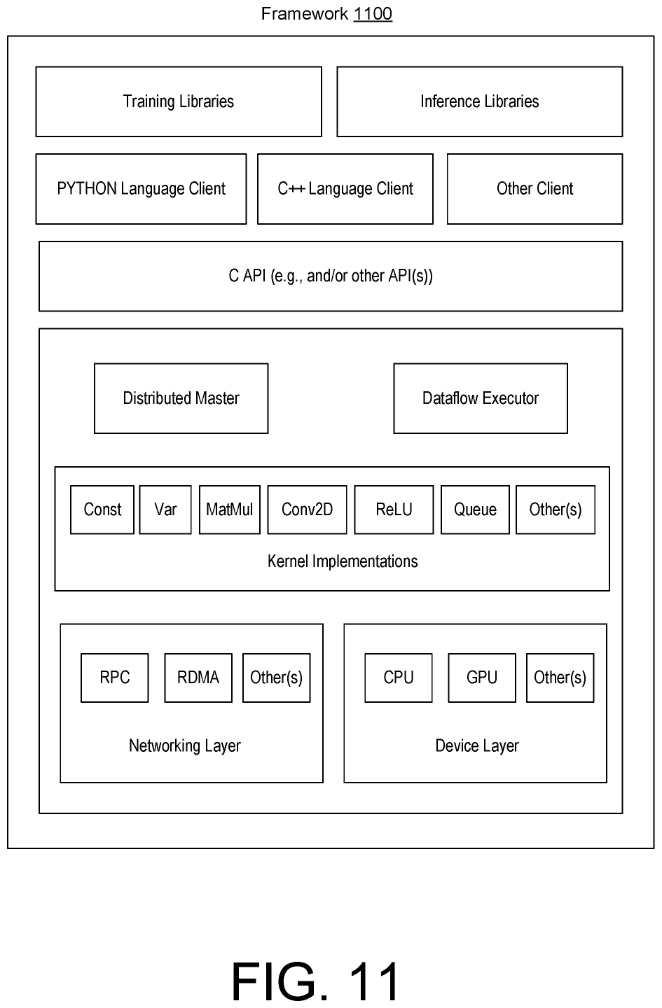

FIG. 11 shows an architecture 1100 of a framework such as the TENSORFLOW framework. As shown, the architecture 1100 includes various features. As an example, in the terminology of the architecture 1100, a client can define a computation as a dataflow graph and, for example, can initiate graph execution using a session. As an example, a distributed master can prune a specific subgraph from the graph, as defined by the arguments to “Session.run ( )”; partition the subgraph into multiple pieces that run in different processes and devices; distributes the graph pieces to worker services; and initiate graph piece execution by worker services. As to worker services (e.g., one per task), as an example, they may schedule the execution of graph operations using kernel implementations appropriate to hardware available (CPUs, GPUs, etc.) and, for example, send and receive operation results to and from other worker services. As to kernel implementations, these may, for example, perform computations for individual graph operations.

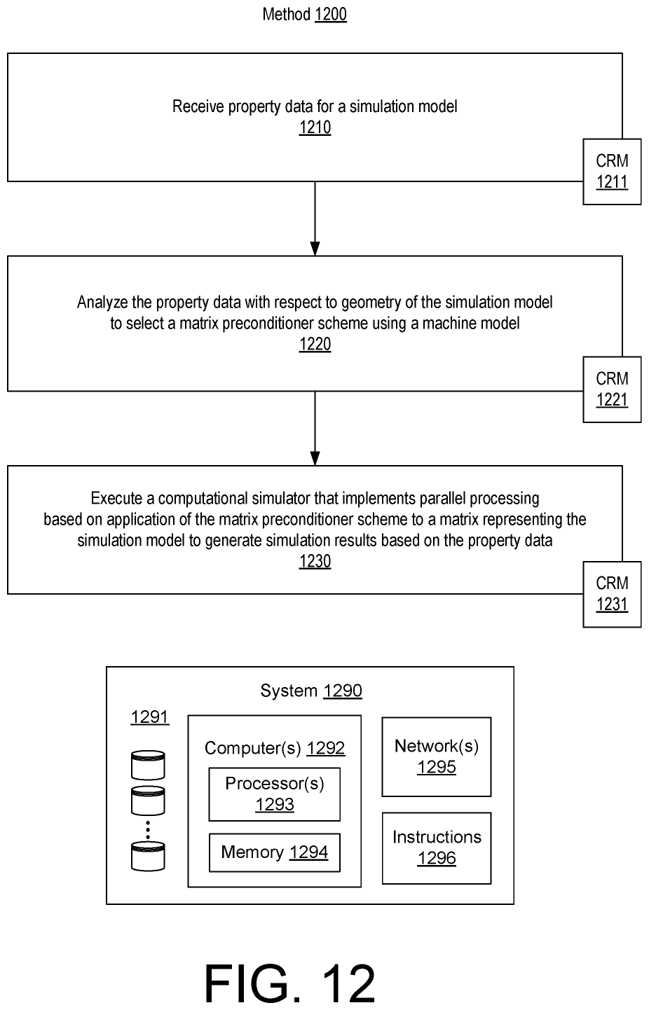

FIG. 12 shows an example of a method 1200 and an example of a system 1290. As shown, the method 1200 includes a reception block 1210 for receiving property data for a simulation model; an analysis block 1220 for analyzing the property data with respect to geometry of the simulation model to select a matrix preconditioner scheme using a machine model; and an execution block 1230 for executing a computational simulator that implements parallel processing based on application of the matrix preconditioner scheme to a matrix representing the simulation model to generate simulation results based on the property data.

The method 1200 is shown as including various computer-readable storage medium (CRM) blocks 1211, 1221, and 1231 that can include processor-executable instructions that can instruct a computing system, which can be a control system, to perform one or more of the actions described with respect to the method 1200.

In the example of FIG. 12, the system 1290 includes one or more information storage devices 1291, one or more computers 1292, one or more networks 1295 and instructions 1296. As to the one or more computers 1292, each computer may include one or more processors (e.g., or processing cores) 1293 and a memory 1294 for storing the instructions 1296, for example, executable by at least one of the one or more processors 1293 (see, e.g., the blocks 1211, 1221 and 1231). As an example, a computer may include one or more network interfaces (e.g., wired or wireless), one or more graphics cards, a display interface (e.g., wired or wireless), etc.

As an example, the method 1200 may be a workflow that can be implemented using one or more frameworks that may be within a framework environment. As an example, the system 1290 can include local and/or remote resources. For example, consider a browser application executing on a client device as being a local resource with respect to a user of the browser application and a cloud-based computing device as being a remote resources with respect to the user. In such an example, the user may interact with the client device via the browser application where information is transmitted to the cloud-based computing device (or devices) and where information may be received in response and rendered to a display operatively coupled to the client device (e.g., via services, APIs, etc.).

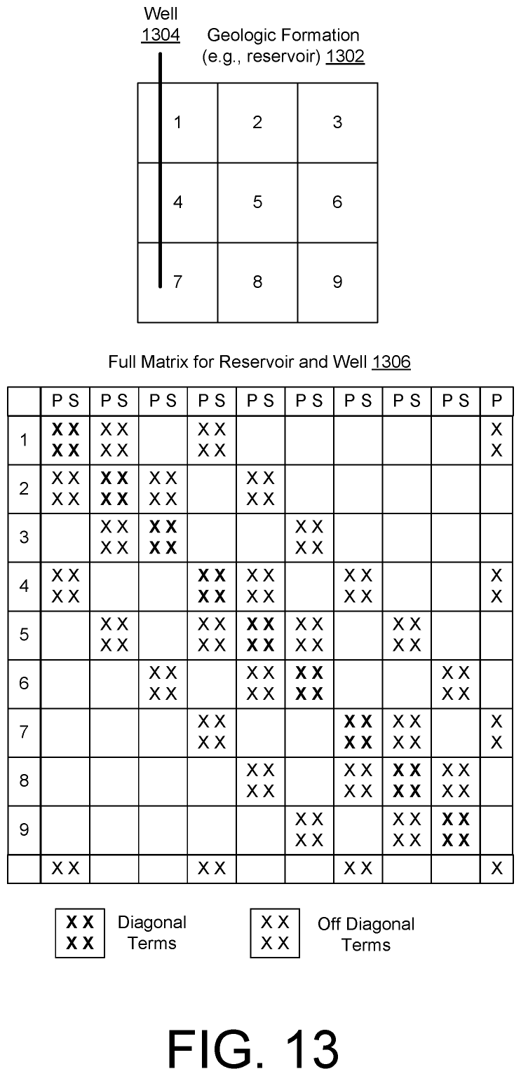

FIG. 13 shows an example of a geologic formation 1302 and a well 1304 and an example of a corresponding matrix 1306 (e.g., a Jacobian matrix of derivatives, etc.). As shown, the geologic formation 1302 is spatially discretized into nine cells that are regularly ordered in space as three layers with three cells in each layer. In the simplistic representation of the geologic formation 1302 of FIG. 13, the cells are numbered 1 to 9 and their corresponding equations for pressure (P) and saturation (S) can be represented readily in the matrix 1306 as diagonal terms and various off diagonal terms that may define a band-width (e.g., a banded matrix). For example, cell 1 has neighboring cells 2 and 4 that result in off diagonal terms (see, e.g., the entries in column 1, rows 2 and 4). In the example of FIG. 13, the presence of the well 1304 introduces additional terms to the system of equations for the geologic formation 1302. As shown, these additional terms appear along a border of the matrix 1306 as associated with cells 1, 4 and 7. Thus, the matrix 1306 includes diagonal terms and various off diagonal terms that may collectively define a band-width as well as border terms that may define a border width.

As explained, characteristics of a spatial grid (e.g., structured, unstructured, etc.) can dictate how equations are setup in a simulator (e.g., matrix size, condition, sparseness, etc.), which may impact computational resource demands, computation time, convergence, etc. As an example, a structured grid may provide a matrix (e.g., a Jacobian matrix) that may be less sparse, more “organized”, etc., when compared to a matrix associated with an unstructured grid. Based on such factors, a reservoir simulator may be configured to perform simulations of geologic formations represented by structured grids.

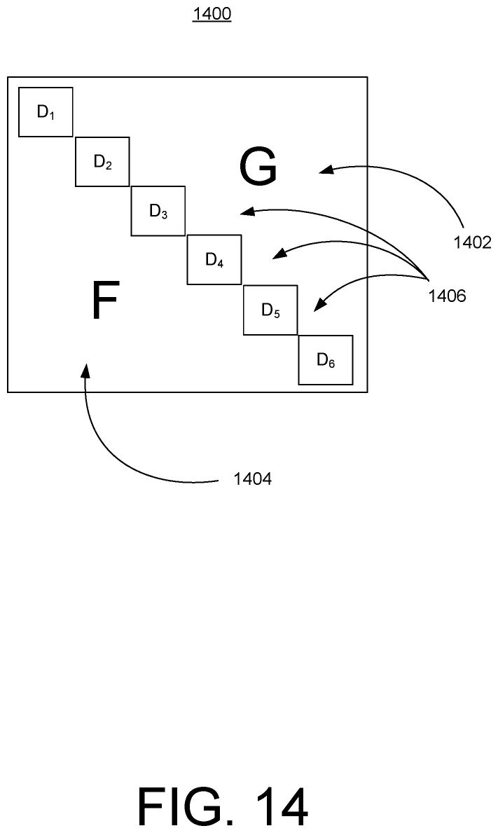

FIG. 14 shows an example of a colored data structure 1400, which is illustrated as a matrix, noting that various forms of vectors, arrays, etc., may be utilized. Specifically, FIG. 14 shows the colored data structure 1400 as a block diagram of a colored matrix. FIG. 14 depicts a matrix (e.g., matrix A) of the coefficients of a linear system with rows and columns reordered based on coloring the unknowns and thereby for parallelism. The matrix A includes sparse matrices 1402 (e.g., G) and 1404 (e.g., F) and diagonal matrices 1406 (e.g., D₁ to D₆).

As an example, the method 1200 of FIG. 12 can provide for selecting a matrix preconditioner scheme amongst a plurality of different matrix preconditioner schemes to generate a color-coded data structure that facilitates parallel processing.

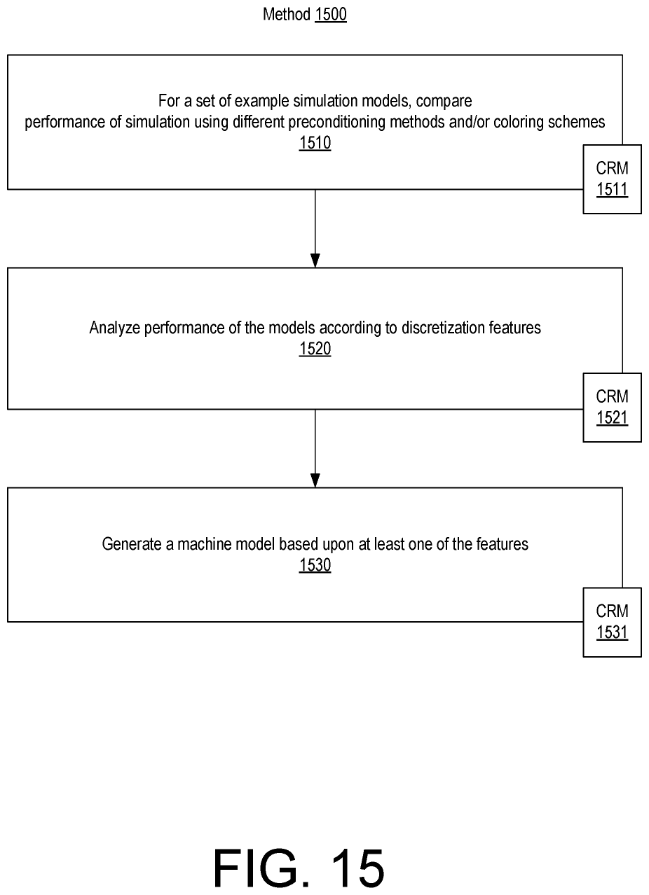

FIG. 15 shows an example of a method 1500 for generating a machine model (e.g., a trained machine model). The method 1500 can include a comparison block 1510 for, using a set of example simulation models comparing the performance of simulation of a simulator using different preconditioning methods (e.g., MC-DILU, MC-ILU, etc.) and/or coloring schemes where each of the schemes may or may not utilize a common number of colors, for example, one type of scheme may be assessed using more than one number of colors (e.g., 16, 32, etc.); an analysis block 1520 for analyzing performance of the models according to discretization features, such as the structure of a grid, the presence of local grid refinements and a ratio of horizontal to vertical permeability or transmissibility where such a ratio may be referred to as an anisotropy ratio; and a generation block 1530 for generating a machine model based upon at least one of the features where such an approach may utilize, for example, one or more threshold values for an anisotropy ratio, etc. As shown in the example of FIG. 5, matrix preconditioner schemes can include General, Strong, Vertical_Strong, and Vertical_Weak where variations thereof may include different numbers of colors.

The method 1500 is shown in FIG. 15 as including various computer-readable storage medium (CRM) blocks 1511, 1521, and 1531 that can include processor-executable instructions that can instruct a computing system, which can be a control system, to perform one or more of the actions described with respect to the method 1500.

As an example, a method can include receiving property data for a simulation model; analyzing the property data with respect to geometry of the simulation model to select a matrix preconditioner scheme using a machine model; and executing a computational simulator that implements parallel processing based on application of the matrix preconditioner scheme to a matrix representing the simulation model to generate simulation results based on the property data. In such an example, the matrix preconditioner scheme can include a specified number of colors where, for example, each of the colors indicates at least one of multiple regions of the matrix amenable to parallel processing.

As an example, a method can utilize a machine model that selects a matrix preconditioner scheme from a plurality of different matrix preconditioner schemes. In such an example, the plurality of matrix preconditioner schemes can include directionally dependent schemes where, for example, one or more of the directionally dependent schemes may differs from another one of the directionally dependent schemes as to a specified number of colors.

As an example, a machine model can assess a simulation model for a structured geometry and/or for an unstructured geometry.

As an example, a machine model can assess a simulation model for a refined geometry and/or for an unrefined geometry.

As an example, a machine model can assess a ratio that depends on at least a portion of property data and at least part of geometry of a simulation model. For example, geometry of a simulation model can be defined at least in part by discretization distances of the simulation model in two orthogonal directions. In such an example, the simulation model can be at least a two-dimensional simulation model, where, for example, one of the discretization distances in one of the two orthogonal directions is less than another one of the discretization distances in another one of the two orthogonal directions.

As an example, a machine model can assess a ratio with respect to at least one threshold value. For example, consider the ratio being an anisotropy ratio that depends at least in part on a material property of a volume of material represented by a simulation model, where the material property is specified by at least a portion of the property data. In such an example, for reservoir simulation, the material property may be permeability or depend on permeability. As an example, an aspect ratio for discretization may be utilized, which may be less than 1, 1 or greater than 1, depending on how discretization is implemented. For example, if cubes are utilized (e.g., consider eight corner nodes), distances between adjacent nodes along orthogonal directions may be equal such that an aspect ratio is 1. Depending on how the ratio is defined, an aspect ratio can differ from one where at least two faces of are rectangular. A cube can be composed of six square faces that meet each other at right angles and as having eight vertices (nodes) and 12 edges. As to hexahedron discretization, consider use of cuboids. A cuboid can be a closed box composed of three pairs of rectangular faces placed opposite each other and joined at right angles to each other and may be referred to as a rectangular parallelepiped. A cuboid can also be a right prism, a special case of the parallelepiped (e.g., a rectangular box).

In various instances, a reservoir model or another type of simulation model can include discretization dimensions or distances that are larger in one direction than in another direction. In such instances, a ratio may exceed 2, may exceed 10, may exceed 100 and may, at times, exceed 10,000. As explained, such dimensions or distances may be utilized to define a mesh-based discretization or a meshless discretization.

As an example, a machine model can include a decision tree structure. For example, consider a decision tree structure that includes leaves, where each of the leaves corresponds to one of a plurality of different matrix preconditioner schemes.

As an example, property data can include permeability data for at least a portion of a reservoir represented by a simulation model. In such an example, a machine model may be utilized to select a matrix preconditioner scheme based at least in part on the permeability data, which may be, for example, anisotropic or geometry dependent.

As an example, a system can include a processor; a memory accessible by the processor; and processor-executable instructions stored in the memory that are executable to instruct the system to: receive property data for a simulation model; analyze the property data with respect to geometry of the simulation model to select a matrix preconditioner scheme using a machine model; and execute a computational simulator that implements parallel processing based on application of the matrix preconditioner scheme to a matrix representing the simulation model to generate simulation results based on the property data.

As an example, one or more non-transitory computer-readable storage media can include computer-executable instructions executable to instruct a computer to: receive property data for a simulation model; analyze the property data with respect to geometry of the simulation model to select a matrix preconditioner scheme using a machine model; and execute a computational simulator that implements parallel processing based on application of the matrix preconditioner scheme to a matrix representing the simulation model to generate simulation results based on the property data.

In an embodiment, a computer program product is provided that includes computer-executable instructions to instruct a computing system to perform the method 1200, the method 1500 or any of the methods described in the foregoing further embodiments.

A computer-readable storage medium (or computer-readable storage media) is non-transitory, not a signal and not a carrier wave. Rather, a computer-readable storage medium is a physical device that can be considered to be circuitry or hardware.

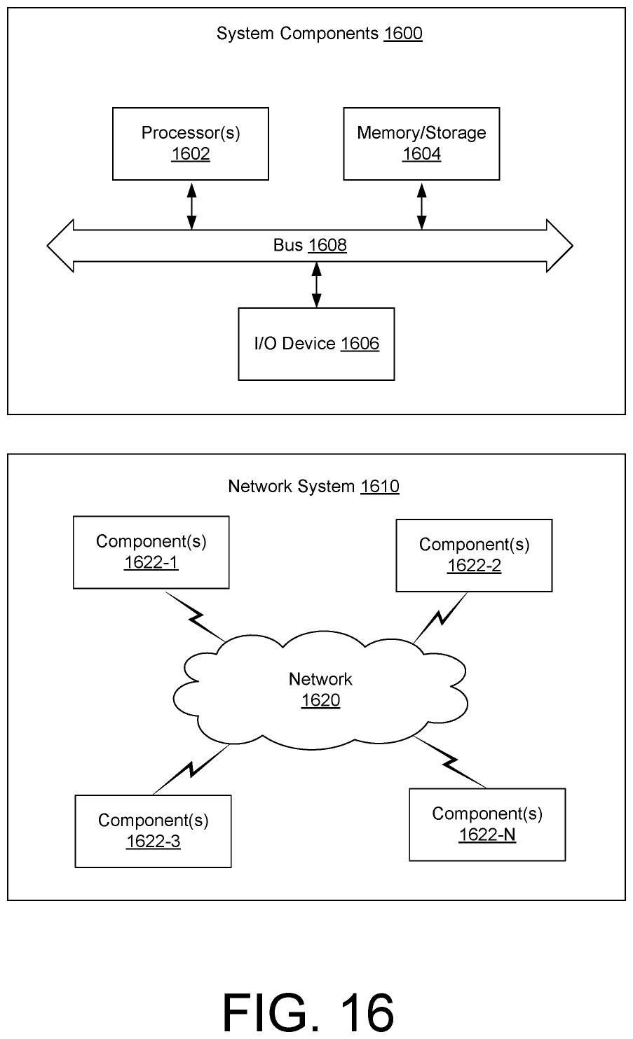

FIG. 16 shows components of an example of a computing system 1600 and an example of a networked system 1610 with a network 1620. The system 1600 includes one or more processors 1602, memory and/or storage components 1604, one or more input and/or output devices 1606 and a bus 1608. In an example embodiment, instructions may be stored in one or more computer-readable media (e.g., memory/storage components 1604). Such instructions may be read by one or more processors (e.g., the processor(s) 1602) via a communication bus (e.g., the bus 1608), which may be wired or wireless. The one or more processors may execute such instructions to implement (wholly or in part) one or more attributes (e.g., as part of a method). A user may view output from and interact with a process via an I/O device (e.g., the device 1606). In an example embodiment, a computer-readable medium may be a storage component such as a physical memory storage device, for example, a chip, a chip on a package, a memory card, etc. (e.g., a computer-readable storage medium).

In an example embodiment, components may be distributed, such as in the network system 1610. The network system 1610 includes components 1622-1, 1622-2, 1622-3, . . . 1622-N. For example, the components 1622-1 may include the processor(s) 1602 while the component(s) 1622-3 may include memory accessible by the processor(s) 1602. Further, the component(s) 1622-2 may include an I/O device for display and optionally interaction with a method. The network may be or include the Internet, an intranet, a cellular network, a satellite network, etc.

As an example, a device may be a mobile device that includes one or more network interfaces for communication of information. For example, a mobile device may include a wireless network interface (e.g., operable via IEEE 802.11, ETSI GSM, BLUETOOTH, satellite, etc.). As an example, a mobile device may include components such as a main processor, memory, a display, display graphics circuitry (e.g., optionally including touch and gesture circuitry), a SIM slot, audio/video circuitry, motion processing circuitry (e.g., accelerometer, gyroscope), wireless LAN circuitry, smart card circuitry, transmitter circuitry, GPS circuitry, and a battery. As an example, a mobile device may be configured as a cell phone, a tablet, etc. As an example, a method may be implemented (e.g., wholly or in part) using a mobile device. As an example, a system may include one or more mobile devices.

As an example, a system may be a distributed environment, for example, a so-called “cloud” environment where various devices, components, etc. interact for purposes of data storage, communications, computing, etc. As an example, a device or a system may include one or more components for communication of information via one or more of the Internet (e.g., where communication occurs via one or more Internet protocols), a cellular network, a satellite network, etc. As an example, a method may be implemented in a distributed environment (e.g., wholly or in part as a cloud-based service).

As an example, information may be input from a display (e.g., consider a touchscreen), output to a display or both. As an example, information may be output to a projector, a laser device, a printer, etc. such that the information may be viewed. As an example, information may be output stereographically or holographically. As to a printer, consider a 2D or a 3D printer. As an example, a 3D printer may include one or more substances that can be output to construct a 3D object. For example, data may be provided to a 3D printer to construct a 3D representation of a subterranean formation. As an example, layers may be constructed in 3D (e.g., horizons, etc.), geobodies constructed in 3D, etc. As an example, holes, fractures, etc., may be constructed in 3D (e.g., as positive structures, as negative structures, etc.).

Although only a few example embodiments have been described in detail above, those skilled in the art will readily appreciate that many modifications are possible in the example embodiments. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures.

What is claimed is:

1. A method comprising:
receiving property data for a simulation model;
analyzing the property data with respect to geometry of the simulation model to select a matrix preconditioner scheme using a machine model; and
executing a computational simulator that implements parallel processing based on application of the matrix preconditioner scheme to a matrix representing the simulation model to generate simulation results based on the property data.

2. The method of claim 1, wherein the matrix preconditioner scheme comprises a specified number of colors.

3. The method of claim 2, wherein each of the colors indicates at least one of multiple regions of the matrix amenable to parallel processing.

4. The method of claim 1, wherein the machine model selects the matrix preconditioner scheme from a plurality of different matrix preconditioner schemes.

5. The method of claim 4, wherein the plurality of matrix preconditioner schemes comprises directionally dependent schemes.

6. The method of claim 5, wherein one of the directionally dependent schemes differs from another one of the directionally dependent schemes as to a specified number of colors.

7. The method of claim 1, wherein the machine model assesses the simulation model for a structured geometry.

8. The method of claim 1, wherein the machine model assesses the simulation model for an unstructured geometry.

9. The method of claim 1, wherein the machine model assesses the simulation model for a refined geometry.

10. The method of claim 1, wherein the machine model assesses the simulation model for an unrefined geometry.

11. The method of claim 1, wherein the machine model assesses a ratio that depends on at least a portion of the property data and at least part of the geometry of the simulation model.

12. The method of claim 11, wherein the geometry of the simulation model is defined at least in part by discretization distances of the simulation model in two orthogonal directions.

13. The method of claim 12, wherein the simulation model comprises at least a two-dimensional simulation model, wherein one of the discretization distances in one of the two orthogonal directions is less than another one of the discretization distances in another one of the two orthogonal directions.

14. The method of claim 11, wherein the machine model assesses the ratio with respect to at least one threshold value.

15. The method of claim 11, wherein the ratio is an anisotropy ratio that depends at least in part on a material property of a volume of material represented by the simulation model, wherein the material property is specified by at least a portion of the property data.

16. The method of claim 1, wherein the machine model comprises a decision tree structure.

17. The method of claim 16, wherein the decision tree structure comprises leaves, wherein each of the leaves corresponds to one of a plurality of different matrix preconditioner schemes.

18. The method of claim 1, wherein the property data comprise permeability data for at least a portion of a reservoir represented by the simulation model.

19. A system comprising:
a processor;
a memory accessible by the processor; and
processor-executable instructions stored in the memory that are executable to instruct the system to:
receive property data for a simulation model;
analyze the property data with respect to geometry of the simulation model to select a matrix preconditioner scheme using a machine model; and
execute a computational simulator that implements parallel processing based on application of the matrix preconditioner scheme to a matrix representing the simulation model to generate simulation results based on the property data.

20. One or more non-transitory computer-readable storage media comprising computer-executable instructions executable to instruct a computer to:
receive property data for a simulation model;
analyze the property data with respect to geometry of the simulation model to select a matrix preconditioner scheme using a machine model; and
execute a computational simulator that implements parallel processing based on application of the matrix preconditioner scheme to a matrix representing the simulation model to generate simulation results based on the property data.
